SEQUENCE LISTING (100 sequences)

SEQ ID NO: 1 - DNA, 21 bp, Artificial sequence
  Synthetic oligonucleotide
tgcatgtttcaccacagtttc

SEQ ID NO: 2 - DNA, 21 bp, Artificial sequence
  Synthetic oligonucleotide
gtagctcacaaccacctgtaa

SEQ ID NO: 3 - DNA, 21 bp, Artificial sequence
  Synthetic oligonucleotide
tccaatgcagtatcccagttc

SEQ ID NO: 4 - DNA, 23 bp, Artificial sequence
  Synthetic oligonucleotide
ccaggacattcaggacatgatta

SEQ ID NO: 5 - DNA, 21 bp, Artificial sequence
  Synthetic oligonucleotide
cccgcagctaattgctttaga

SEQ ID NO: 6 - DNA, 23 bp, Artificial sequence
  Synthetic oligonucleotide
cataaacagacctctggacaacc

SEQ ID NO: 7 - DNA, 20 bp, Artificial sequence
  Synthetic oligonucleotide
ctgccagatgaccttgagtg

SEQ ID NO: 8 - DNA, 22 bp, Artificial sequence
  Synthetic oligonucleotide
gccctgtagtttatcaccttgt

SEQ ID NO: 9 - DNA, 24 bp, Artificial sequence
  Synthetic oligonucleotide
caaagggaattatgttctgggaaa

SEQ ID NO: 10 - DNA, 23 bp, Artificial sequence
  Synthetic oligonucleotide
ggaaagagtaaggtctcggtatg

SEQ ID NO: 11 - DNA, 18 bp, Artificial sequence
  Synthetic oligonucleotide
cccacacacctgtaaccc

SEQ ID NO: 12 - DNA, 21 bp, Artificial sequence
  Synthetic oligonucleotide
tgctgaaagctgagaccatac

SEQ ID NO: 13 - DNA, 20 bp, Artificial sequence
  Synthetic oligonucleotide
catttattatcgcggcccta

SEQ ID NO: 14 - DNA, 20 bp, Artificial sequence
  Synthetic oligonucleotide
tgttgggttgtttgatcctg

SEQ ID NO: 15 - DNA, 20 bp, Artificial sequence
  Synthetic oligonucleotide
tgctagccgcaggcattact

SEQ ID NO: 16 - DNA, 21 bp, Artificial sequence
  Synthetic oligonucleotide
cgggatcaaagaaagttgtgt

SEQ ID NO: 17 - DNA, 18 bp, Artificial sequence
  Synthetic oligonucleotide
cagggccatctcagaacc

SEQ ID NO: 18 - DNA, 20 bp, Artificial sequence
  Synthetic oligonucleotide
gctattggaaagcctcttgc

SEQ ID NO: 19 - DNA, 20 bp, Artificial sequence
  Synthetic oligonucleotide
gaagcgattctagggagcag

SEQ ID NO: 20 - DNA, 21 bp, Artificial sequence
  Synthetic oligonucleotide
ggagcagcgattctgagtaga

SEQ ID NO: 21 - DNA, 21 bp, Artificial sequence
  Synthetic oligonucleotide
ctcagctctctacccaaacgc

SEQ ID NO: 22 - DNA, 21 bp, Artificial sequence
  Synthetic oligonucleotide
ccgcttggtgatctcacactc

SEQ ID NO: 23 - DNA, 20 bp, Artificial sequence
  Synthetic oligonucleotide
cagatcgggagcttgtgttc

SEQ ID NO: 24 - DNA, 20 bp, Artificial sequence
  Synthetic oligonucleotide
tggtccccaagtgtcagact

SEQ ID NO: 25 - DNA, 23 bp, Artificial sequence
  Synthetic oligonucleotide
gaatctttttccctgcactacga

SEQ ID NO: 26 - DNA, 23 bp, Artificial sequence
  Synthetic oligonucleotide
gctggaatcaatgtgtcgatctt

SEQ ID NO: 27 - DNA, 20 bp, Artificial sequence
  Synthetic oligonucleotide
gcttccaggccatattggag

SEQ ID NO: 28 - DNA, 19 bp, Artificial sequence
  Synthetic oligonucleotide
gggggcatgacctcatctt

SEQ ID NO: 29 - DNA, 21 bp, Artificial sequence
  Synthetic oligonucleotide
gaggtcactcctatcctctgg

SEQ ID NO: 30 - DNA, 22 bp, Artificial sequence
  Synthetic oligonucleotide
gccatttcctccgacttttctc

SEQ ID NO: 31 - DNA, 23 bp, Artificial sequence
  Synthetic oligonucleotide
ggacaattttccaaccaatctgc

SEQ ID NO: 32 - DNA, 21 bp, Artificial sequence
  Synthetic oligonucleotide
tcggttcattccctctgcata

SEQ ID NO: 33 - DNA, 21 bp, Artificial sequence
  Synthetic oligonucleotide
tcccagcttgattcctctctt

SEQ ID NO: 34 - DNA, 21 bp, Artificial sequence
  Synthetic oligonucleotide
gggttgttctttctgtcccag

SEQ ID NO: 35 - DNA, 22 bp, Artificial sequence
  Synthetic oligonucleotide
ctgaataagtgcggacctatgg

SEQ ID NO: 36 - DNA, 22 bp, Artificial sequence
  Synthetic oligonucleotide
agtattgcctccgttgatgttc

SEQ ID NO: 37 - DNA, 19 bp, Artificial sequence
  Synthetic oligonucleotide
gccgctgtctgttccattc

SEQ ID NO: 38 - DNA, 23 bp, Artificial sequence
  Synthetic oligonucleotide
gcatcaatgtctggcttgttgaa

SEQ ID NO: 39 - DNA, 21 bp, Artificial sequence
  Synthetic oligonucleotide
acgtccagttcgatgagggat

SEQ ID NO: 40 - DNA, 21 bp, Artificial sequence
  Synthetic oligonucleotide
tttctggcctctaaccaagcc

SEQ ID NO: 41 - DNA, 20 bp, Artificial sequence
  Synthetic oligonucleotide
gcacaccaggcagtagcttt

SEQ ID NO: 42 - DNA, 23 bp, Artificial sequence
  Synthetic oligonucleotide
caggagttgattccagacaggta

SEQ ID NO: 43 - DNA, 19 bp, Artificial sequence
  Synthetic oligonucleotide
cagcacagcatcgtaccca

SEQ ID NO: 44 - DNA, 21 bp, Artificial sequence
  Synthetic oligonucleotide
tcccaatgccgttctcaaaat

SEQ ID NO: 45 - DNA, 20 bp, Artificial sequence
  Synthetic oligonucleotide
gacgagccgaaacccatcag

SEQ ID NO: 46 - DNA, 19 bp, Artificial sequence
  Synthetic oligonucleotide
agtcggaccttgaccgtgt

SEQ ID NO: 47 - DNA, 23 bp, Artificial sequence
  Synthetic oligonucleotide
agggtttagttttgagttgacgg

SEQ ID NO: 48 - DNA, 21 bp, Artificial sequence
  Synthetic oligonucleotide
ccccgcttttgtcatattccg

SEQ ID NO: 49 - DNA, 21 bp, Artificial sequence
  Synthetic oligonucleotide
agcctgtagctcactgttgtc

SEQ ID NO: 50 - DNA, 22 bp, Artificial sequence
  Synthetic oligonucleotide
atgtactgaaagttagcacccg

SEQ ID NO: 51 - DNA, 20 bp, Artificial sequence
  Synthetic oligonucleotide
tgcatttgccgcagctttac

SEQ ID NO: 52 - DNA, 20 bp, Artificial sequence
  Synthetic oligonucleotide
gttggcccagatttcgttca

SEQ ID NO: 53 - DNA, 22 bp, Artificial sequence
  Synthetic oligonucleotide
ccaagcacattacgaccatttc

SEQ ID NO: 54 - DNA, 20 bp, Artificial sequence
  Synthetic oligonucleotide
ccgtggaccagcgtatagga

SEQ ID NO: 55 - DNA, 23 bp, Artificial sequence
  Synthetic oligonucleotide
ccacagaacagctacccaaattt

SEQ ID NO: 56 - DNA, 20 bp, Artificial sequence
  Synthetic oligonucleotide
tccacagggctgcaattttc

SEQ ID NO: 57 - DNA, 21 bp, Artificial sequence
  Synthetic oligonucleotide
tgcaatcttcggccagttact

SEQ ID NO: 58 - DNA, 23 bp, Artificial sequence
  Synthetic oligonucleotide
ctcggatgctattcgatcaagtt

SEQ ID NO: 59 - DNA, 20 bp, Artificial sequence
  Synthetic oligonucleotide
agaactggacccggttacca

SEQ ID NO: 60 - DNA, 20 bp, Artificial sequence
  Synthetic oligonucleotide
cacttcgctgatacccctga

SEQ ID NO: 61 - DNA, 22 bp, Artificial sequence
  Synthetic oligonucleotide
tggaaaatggttcgagagtcag

SEQ ID NO: 62 - DNA, 23 bp, Artificial sequence
  Synthetic oligonucleotide
cattccgtctctaggttaaagcg

SEQ ID NO: 63 - DNA, 23 bp, Artificial sequence
  Synthetic oligonucleotide
caggaattgttacggttccctaa

SEQ ID NO: 64 - DNA, 22 bp, Artificial sequence
  Synthetic oligonucleotide
cctgaattaacttgtcccgtga

SEQ ID NO: 65 - DNA, 20 bp, Artificial sequence
  Synthetic oligonucleotide
gcccagtacctccgaaagtc

SEQ ID NO: 66 - DNA, 23 bp, Artificial sequence
  Synthetic oligonucleotide
gccttaacatactcctccttgtc

SEQ ID NO: 67 - DNA, 21 bp, Artificial sequence
  Synthetic oligonucleotide
ctggattctggcgatggtgta

SEQ ID NO: 68 - DNA, 21 bp, Artificial sequence
  Synthetic oligonucleotide
cggacaatttcacgttcagca

SEQ ID NO: 69 - DNA, 21 bp, Artificial sequence
  Synthetic oligonucleotide
tctgccaactaccgagcctat

SEQ ID NO: 70 - DNA, 21 bp, Artificial sequence
  Synthetic oligonucleotide
ctcttctgcctctcgttccat

SEQ ID NO: 71 - DNA, 21 bp, Artificial sequence
  Synthetic oligonucleotide
cagaggaggccaacgtagaag

SEQ ID NO: 72 - DNA, 20 bp, Artificial sequence
  Synthetic oligonucleotide
ctccatcggggatcttgggt

SEQ ID NO: 73 - DNA, 21 bp, Artificial sequence
  Synthetic oligonucleotide
gcggtagaacagttgacagag

SEQ ID NO: 74 - DNA, 19 bp, Artificial sequence
  Synthetic oligonucleotide
ccagctccttggtgctgat

SEQ ID NO: 75 - DNA, 21 bp, Artificial sequence
  Synthetic oligonucleotide
gagaacgctcacacaaagacc

SEQ ID NO: 76 - DNA, 19 bp, Artificial sequence
  Synthetic oligonucleotide
caattcgttggagccccat

SEQ ID NO: 77 - DNA, 21 bp, Artificial sequence
  Synthetic oligonucleotide
aaagtgcaatacctcactcgc

SEQ ID NO: 78 - DNA, 22 bp, Artificial sequence
  Synthetic oligonucleotide
ggcatttcaatagtggaggctc

SEQ ID NO: 79 - DNA, 21 bp, Artificial sequence
  Synthetic oligonucleotide
acacccagtggctataccctg

SEQ ID NO: 80 - DNA, 21 bp, Artificial sequence
  Synthetic oligonucleotide
ccgtaggatgcttcatcaccc

SEQ ID NO: 81 - DNA, 23 bp, Artificial sequence
  Synthetic oligonucleotide
ctgtttaagactcaccctgagac

SEQ ID NO: 82 - DNA, 20 bp, Artificial sequence
  Synthetic oligonucleotide
ggtgcaaccatgcttcttca

SEQ ID NO: 83 - DNA, 21 bp, Artificial sequence
  Synthetic oligonucleotide
acctcatcgtggttgtagcct

SEQ ID NO: 84 - DNA, 22 bp, Artificial sequence
  Synthetic oligonucleotide
ggatacccctgatagctgatgt

SEQ ID NO: 85 - DNA, 21 bp, Artificial sequence
  Synthetic oligonucleotide
gtgctgcctgagtataagctg

SEQ ID NO: 86 - DNA, 20 bp, Artificial sequence
  Synthetic oligonucleotide
ccgagtacggtgtgaagact

SEQ ID NO: 87 - DNA, 21 bp, Artificial sequence
  Synthetic oligonucleotide
atgggcagtgtgagaaccaac

SEQ ID NO: 88 - DNA, 22 bp, Artificial sequence
  Synthetic oligonucleotide
tggactttactcttgccattcc

SEQ ID NO: 89 - DNA, 22 bp, Artificial sequence
  Synthetic oligonucleotide
atggcaaacttcctgttacctc

SEQ ID NO: 90 - DNA, 20 bp, Artificial sequence
  Synthetic oligonucleotide
ccacgggcttgtttttcagc

SEQ ID NO: 91 - DNA, 21 bp, Artificial sequence
  Synthetic oligonucleotide
ggaaccgaggcaatactcagg

SEQ ID NO: 92 - DNA, 23 bp, Artificial sequence
  Synthetic oligonucleotide
ccgttaatagcgtagatggtggt

SEQ ID NO: 93 - DNA, 19 bp, Artificial sequence
  Synthetic oligonucleotide
cctgctggtggttagcgtg

SEQ ID NO: 94 - DNA, 21 bp, Artificial sequence
  Synthetic oligonucleotide
tctgcctccgtctgtttagaa

SEQ ID NO: 95 - DNA, 2293 bp, Homo sapiens
  CDS (235)..(1506)
tagaggtctcccgcgggcggggagggggaggcgtagcaactttaggcaacttcccaaaggtgtgcgcaggttgggggcgggacgcggcgccccgggaggtggcggcctctgcgacagcgggagtataagagtggacctgcaggctggtcgcgaggaggtggagcggcgcccgccgtgtgcctgggaccggcatgctggggcaggagggcagccgcgtgtcaggtgaccagcgccatgtccagccaggtggtgggcattgagcctctctacatcaaggcagagccggccagccctgacagtccaaagggttcctcggagacagagaccgagcctcctgtggccctggcccctggtccagctcccactcgctgcctcccaggccacaaggaagaggaggatggggagggggctgggcctggcgagcagggcggtgggaagctggtgctcagctccctgcccaagcgcctctgcctggtctgtggggacgtggcctccggctaccactatggtgtggcatcctgtgaggcctgcaaagccttcttcaagaggaccatccaggggagcatcgagtacagctgtccggcctccaacgagtgtgagatcaccaagcggagacgcaaggcctgccaggcctgccgcttcaccaagtgcctgcgggtgggcatgctcaaggagggagtgcgcctggaccgcgtccggggtgggcggcagaagtacaagcggcggccggaggtggacccactgcccttcccgggccccttccctgctgggcccctggcagtcgctggaggcccccggaagacagcagccccagtgaatgcactggtgtctcatctgctggtggttgagcctgagaagctctatgccatgcctgaccccgcaggccctgatgggcacctcccagccgtggctaccctctgtgacctctttgaccgagagattgtggtcaccatcagctgggccaagagcatcccaggcttctcatcgctgtcgctgtctgaccagatgtcagtactgcagagcgtgtggatggaggtgctggtgctgggtgtggcccagcgctcactgccactgcaggatgagctggccttcgctgaggacttagtcctggatgaagagggggcacgggcagctggcctgggggaactgggggctgccctgctgcaactagtgcggcggctgcaggccctgcggctggagcgagaggagtatgttctactaaaggccttggcccttgccaattcagactctgtgcacatcgaagatgccgaggctgtggagcagctgcgagaagctctgcacgaggccctgctggagtatgaagccggccgggctggccccggagggggtgctgagcggcggcgggcgggcaggctgctgctcacgctaccgctcctccgccagacagcgggcaaagtgctggcccatttctatggggtgaagctggagggcaaggtgcccatgcacaagctgttcttggagatgctcgaggccatgatggactgaggcaaggggtgggactggtgggggttctggcaggacctgcctagcatggggtcagccccaagggctggggcggagctggggtctgggcagtgccacagcctgctggcagggccagggcaatgccatcagcccctgggaacaggccccacgccctctcctccccctcctagggggtgtcagaagctgggaacgtgtgtccaggctctgggcacagtgctgccccttgcaagccataacgtgcccccagagtgtagggggccttgcggaagccatagggggctgcacgggatgcgtgggaggcagaaacctatctcagggagggaaggggatggaggccagagtctcccagtgggtgatgcttttgctgctgcttaatcctaccccctcttcaaagcagagtgggacttggagagcaaaggcccatgcccccttcgctcctcctctcatcatttgcattgggcattagtgtccccccttgaagcaataactccaagcagactccagcccctggacccctggggtggccagggcttccccatcagctcccaacgagcctcctcagggggtaggagagcactgcctctatgccctgcagagcaataacactatatttatttttgggtttggccagggaggcgcagggacatggggcaagccagggcccagagcccttggctgtacagagactctattttaatgtatatttgctgcaaagagaaaccgcttttggttttaaacctttaatgagaaaaaaatatataataccgagctcaaaaaaaaaaaaaaa

SEQ ID NO: 96 - protein, 423 aa, Homo sapiens
MSSQVVGIEPLYIKAEPASPDSPKGSSETETEPPVALAPGPAPTRCLPGHKEEEDGEGAGPGEQGGGKLVLSSLPKRLCLVCGDVASGYHYGVASCEACKAFFKRTIQGSIEYSCPASNECEITKRRRKACQACRFTKCLRVGMLKEGVRLDRVRGGRQKYKRRPEVDPLPFPGPFPAGPLAVAGGPRKTAAPVNALVSHLLVVEPEKLYAMPDPAGPDGHLPAVATLCDLFDREIVVTISWAKSIPGFSSLSLSDQMSVLQSVWMEVLVLGVAQRSLPLQDELAFAEDLVLDEEGARAAGLGELGAALLQLVRRLQALRLEREEYVLLKALALANSDSVHIEDAEAVEQLREALHEALLEYEAGRAGPGGGAERRRAGRLLLTLPLLRQTAGKVLAHFYGVKLEGKVPMHKLFLEMLEAMMD

SEQ ID NO: 97 - DNA, 5336 bp, Homo sapiens
  CDS (319)..(1626)
ggagggcgagggcgggctcctctggaggcggcgggctccaggccgggctccctaaacgggtcagcaattggagcgggaaggccggacagagacagggtcttgctttgtcacccaggctggagtgcagtgccacaatcagagcttactcctgccttgaactcctgggcttcagggatcctgctgcctcggccctccaagcagctgagactacagagctgacaatttactggttcatcaatgaaacaatattaaattatgaagatgtaaggaaaaaatcctacgctaacactgtcgcagtttgaaaggcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagtttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccatactgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagaggcaataaggcaaatgccagaattaaaaaaaaaaatcatcaaagacagaaaatgcctgaccaaattctaaaacctaatccatataagtttattcatttaggaatgttcgtttaaattaatctgcagtttttaccaagagctaagccaatatatgtgcttttcaaccagtattgtcacagcatgaaagtcaagtcaggttccagactgttaagaggtgtaatctaatgaagaaatcaattagatgccccgaaatctacagtcgctgaataaccaataaacagtaacctccatcaaatgctataccaatggaccagtgttagtagctgctccctgtattatgtgaacagtcttattctatgtacacagatgtaattaaaattgtaatcctaacaaacaaaagaaatgtagttcagcttttcaatgtttcatgtttgctgtgcttttctgaattttatgttgcattcaaagactgttgtcttgttcttgtggtgtttggattcttgtggtgtgtgcttttagacacagggtagaattagagacaatattggatgtacaattcctcaggagactacagtagtatattctattccttaccagtaataaggttcttcctaataataattaagagattgaaactccaaacaagtattcattatgaacagatacacatcaaaatcataataatattttcaaaacaaggaataatttctctaatggtttattatagaataccaatgtatagcttagaaataaaactttgaatatttcaagaatatagataagtctaatttttaaatgctgtatatatggctttcactcaatcatctctcagatgttgttattaactcgctctgtgttgttgcaaaactttttggtgcagattcgtttccaaaactattgctactttgtgtgctttaaacaaaataccttgggttgatgaaacatcaacccagtgctaggaatactgtgtatctatcattagctatatgggactatattgtagattgtggtttctcagtagagaagtgactgtagtgtgattctagataaatcatcattagcaattcattcagatggtcaataacttgaaatttatagctgtgataggagttcagaaattggcacatccctttaaaaataacaacagaaaatacaactcctgggaaaaaaggtgctgattctataagattatttatatatgtaagtgtttaaaaagattattttccagaaagtttgtgcagggtttaagttgctactattcaactacactatatataaataaaatatatacaatatatacattgttttcactgtatcacattaaagtacttgggcttcagaagtaagagccaaccaactgaaaacctgagatggagatatgttcaaagaatgagatacaattttttagttttcagtttaagtaactctcagcattacaaaagagtaagtatctcacaaataggaaataaaactaaaacgtggatttaaaaagaactgcacgggctttagggtaaatgctcatcttaaacctcactagagggaagtcttctcaagtttcaagcaagaccatttacttaatgtgaagttttggaaagttataaaggtgtatgttttagccatatgattttaattttaattttgcttcttttaggttcgttcttatttaaagcaatatgattgtgtgactccttgtagttacacttgtgtttcaatcagatcagattgttgtatttattccactattttgcatttaaatgataacataaaagatataaaaaatttaaaactgctatttttcttatagaagagaaaatgggtgttggtgattgtattttaattatttaagcgtctctgtttacctgcctaggaaaacattttatggcagtcttatgtgcaaagatcgtaaaaggacaaaaaatttaaactgcttataataatccaggagttgcattatagccagtagtaaaaataataataataataataaaaccatgtctatagctgtagatgggcttcacatctgtaaagcaatcaattgtatatttttgtgatgtgtaccatactgtgtgctccagcaaatgtccatttgtgtaaatgtatttattttatattgtatatattgttaaatgcaaaaaggagatatgattctgtaactccaatcagttcagatgtgtaactcaaattattatgcctttcaggatgatggtagagcaatattaaacaagcttccacttttgactgctaaaaaaaaaaaaaaaaa

SEQ ID NO: 98 - protein, 435 aa, Homo sapiens
MSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 99 - DNA, 3527 bp, Homo sapiens
  CDS (127)..(2352)
agtgaccgccctttgccactccccctgcctcctctccgcctttaacttctcgggaagatgaggcagtttggcatctgtggccgagttgctgttgccgggtgatagttggagcggagacttagcataatggcagaacctgtttctccactgaagcactttgtgctggctaagaaggcgattactgcaatctttgaccagttactggagtttgttactgaaggatcacattttgttgaagcaacatataagaatccggaacttgatcgaatagccactgaagatgatctggtagaaatgcaaggatataaagacaagctttccatcattggtgaggtgctatctcggagacacatgaaggtggcattttttggcaggacaagcagtgggaagagctctgttatcaatgcaatgttgtgggataaagttctccctagtgggattggccatataaccaattgcttcctaagtgttgaaggaactgatggagataaagcctatcttatgacagaaggatcagatgaaaaaaagagtgtgaagacagttaatcaactggcccatgcccttcacatggacaaagatttgaaagctggctgtcttgtacgtgtgttttggccaaaagcaaaatgtgccctcttgagagatgacctggtgttagtagacagtccaggcacagatgtcactacagagctggatagctggattgataagttttgcctagatgctgatgtctttgttttggtcgcaaactctgaatcaacactaatgaatacggaaaaacacttttttcacaaggtgaatgagcggctttccaagcctaatattttcattctcaataatcgttgggatgcctctgcatcagagccagaatatatggaagacgtacgcagacagcacatggaaagatgcctgcatttcttggtggaggagctcaaagttgtaaatgctttagaagcacagaatcgtatcttctttgtttcagcaaaggaagttcttagtgctagaaagcaaaaagcacaggggatgccagaaagtggtgtggcacttgctgaaggatttcatgcaagattacaggaatttcagaattttgaacaaatctttgaggagtgtatctcgcagtcagcagtgaaaacaaagttcgaacagcacactatcagagctaaacagatactagctactgtgaaaaacataatggattcagtaaacctggcagctgaagataaaaggcattattcagtggaagagagggaagaccaaattgatagactggactttattcgaaaccagatgaaccttttaacactggatgttaagaaaaaaatcaaggaggttaccgaggaggtggcaaacaaagtttcatgtgcaatgacagatgaaatttgtcgactgtctgttttggttgatgaattttgttcagagtttcatcctaatccagatgtattaaaaatatataaaagtgaattaaataagcacatagaggatggtatgggaagaaatttggctgatcgatgcaccgatgaagtaaacgccttagtgcttcagacccagcaagaaattattgaaaatttgaagccattacttccagctggtatacaggataaactacatacactgatcccttgcaagaaatttgatctcagttataatctaaattaccacaagttatgttcagattttcaagaggatattgtatttcgtttttccctgggctggtcttcccttgtacatcgatttttgggccctagaaatgctcaaagggtgctcctaggattatcagagcctatctttcagctccctagatctttagcttctactcccactgctcctaccactccagcaacgccagataatgcatcacaggaagaactcatgattacattagtaacaggattggcgtccgttacatctagaacttctatgggcatcattattgttggaggagtgatttggaaaactataggctggaaactcctatctgtttcattaactatgtatggagctttgtatctttatgaaagactgagctggaccacccatgccaaggagcgagcctttaaacagcagtttgtaaactatgcaactgaaaaactgaggatgattgttagctccacgagtgcaaactgcagtcaccaagtaaaacaacaaatagctaccacttttgctcgcctgtgccaacaagttgatattactcaaaaacagctggaagaagaaattgctagattacccaaagaaatagatcagttggagaaaatacaaaacaattcaaagctcttaagaaataaagctgttcaacttgaaaatgagctggagaattttactaagcagtttctaccttcaagcaatgaagaatcctaacaatagagattgctttggtgaccatgataggaggaaacgaaacttgtaagattggaacagttgttatttttatgaaattactttaaatatgaattgtactaactgtacctaaatagcaaagccctgtgtagattctggtaatgatctgtctcagggtatgtgtatttttgaagagtgttatgtccttagttttaattttgagtaaagaaaaggctaaaatcatgaattagttacaagcaacagtaccaacttatgtgacccctgaggggtggggctgtgagctcttaatttgtttttgattctgaaaaactctgcttcctggcatccaggagttagagattgagcctttcatcttctttctcaaaactagtttttgatgctttctttcatgggaatagtcacttttttatttagtaaatcgcattgctggaaccaccaaggagtgtggaatgtccttgagtgtattatttatgcaagtcacagtcacgttgccatcatggcagctatgtgaaacactaataaatgtgtttttactttttattcccgttaaaactgatgtaaaacaggataaaggcttgttatagtcacttataagtatctgggtctaagtaatttccttagatgtttctaaagaaacattttcagctttgctcccattatgattccaataaggaacgctttcctagtgcaattttaggagtaaagtttgaagagataaaaatagccaaagataggagacgtctgaattttgaatgataaacagtgatgttttaaaaaagctgttgttcttcaggaggcatttgcctaggatattgctggattataccccattggaggcttttaattttatttgtatgaattttccaggatttcattaaaaattattattgtattttttaccttaatgaaagattttgggttcaaatatctttctatattaaaagctgattgagtctgtacatatgtaaattatgcctagtggaggttctgttgactttcttccccactgtggaagaggccagttttgcctccatttgcacattcatttcagttatttctgatccataaatataacatttacaaaattcttccttgagctggtggaaatgcctcaccagtttcctctttaatgaatcaaataaaatctttaactgatgttaaaaaaaaaaaaaaaa

SEQ ID NO: 100 - protein, 741 aa, Homo sapiens
MAEPVSPLKHFVLAKKAITAIFDQLLEFVTEGSHFVEATYKNPELDRIATEDDLVEMQGYKDKLSIIGEVLSRRHMKVAFFGRTSSGKSSVINAMLWDKVLPSGIGHITNCFLSVEGTDGDKAYLMTEGSDEKKSVKTVNQLAHALHMDKDLKAGCLVRVFWPKAKCALLRDDLVLVDSPGTDVTTELDSWIDKFCLDADVFVLVANSESTLMNTEKHFFHKVNERLSKPNIFILNNRWDASASEPEYMEDVRRQHMERCLHFLVEELKVVNALEAQNRIFFVSAKEVLSARKQKAQGMPESGVALAEGFHARLQEFQNFEQIFEECISQSAVKTKFEQHTIRAKQILATVKNIMDSVNLAAEDKRHYSVEEREDQIDRLDFIRNQMNLLTLDVKKKIKEVTEEVANKVSCAMTDEICRLSVLVDEFCSEFHPNPDVLKIYKSELNKHIEDGMGRNLADRCTDEVNALVLQTQQEIIENLKPLLPAGIQDKLHTLIPCKKFDLSYNLNYHKLCSDFQEDIVFRFSLGWSSLVHRFLGPRNAQRVLLGLSEPIFQLPRSLASTPTAPTTPATPDNASQEELMITLVTGLASVTSRTSMGIIIVGGVIWKTIGWKLLSVSLTMYGALYLYERLSWTTHAKERAFKQQFVNYATEKLRMIVSSTSANCSHQVKQQIATTFARLCQQVDITQKQLEEEIARLPKEIDQLEKIQNNSKLLRNKAVQLENELENFTKQFLPSSNEES

(19) United States
(12) Patent Application Publication
(10) Pub. No.: US 2018/0015057 A1
(43) Pub. Date: Jan. 18, 2018

(54) ERR ALPHA AND ERR GAMMA ARE ESSENTIAL COORDINATORS OF CARDIAC METABOLISM AND FUNCTION

(71) Applicant: Salk Institute for Biological Studies, La Jolla, CA (US)

(72) Inventors: Ronald M. Evans, La Jolla, CA (US); Liming Pei, Philadelphia, PA (US); Michael Downes, San Diego, CA (US); Ruth T. Yu, La Jolla, CA (US); Annette Atkins, San Diego, CA (US)

(73) Assignee: Salk Institute for Biological Studies, La Jolla, CA (US)

(21) Appl. No.: 15/709,292

(22) Filed: Sep. 19, 2017

Related U.S. Application Data
(63) Continuation of application No. PCT/US2016/025568, filed on Apr. 1, 2016.
(60) Provisional application No. 62/142,323, filed on Apr. 2, 2015.

(51) Int. Cl. A61K 31/166; A61K 31/415; A61K 31/40; A61K 31/44; A61K 31/519; A61K 45/06; A61K 48/00

(52) U.S. Cl. CPC A61K 31/166; A61K 31/519; A61K 31/415; A61K 45/06; A61K 31/40; A61K 31/44; A61K 48/00; A61K 2300/00

(57) ABSTRACT

Provided herein are methods and kits for increasing cardiac contraction, increasing mitochondrial activity, and/or increasing oxphos activity. Such methods include use of therapeutically effective amounts of one or more agents that increases estrogen-related receptor (ERR) α activity and one or more agents that increases ERRγ activity. In some examples, the method further includes administering a therapeutically effective amount of one or more agents that increases mitofusin 1 (Mfn1) activity.

### CROSS REFERENCE TO RELATED APPLICATIONS

This application is a continuation of International Application No. PCT/US2016/025568, filed on Apr. 1, 2016, which was published in English under PCT Article 21(2), which in turn which claims priority to U.S. Provisional Application No. 62/142,323, filed on Apr. 2, 2015, both herein incorporated by reference.

### ACKNOWLEDGMENT OF GOVERNMENT SUPPORT

This invention was made with government support under grant numbers HL105734, HL105278 and DK057978 awarded by the National Institutes of Health. The government has certain rights in the invention.

### FIELD

This application provides methods and kits for increasing cardiac contraction, increasing mitochondrial activity, and/or increasing oxphos activity, by using therapeutically effective amounts of one or more agents that increases estrogen-related receptor (ERR) α activity and one or more agents that increases ERRγ activity.

### BACKGROUND

Every cell's own survival and vital functions are supported by the energy-generating metabolic pathways. The cellular energy supply and demand must be coordinated and an imbalance results in cellular dysfunctions and diseases from heart failure to obesity. Although the regulation of cellular energy production and consumption individually are the focus of intensive research, little understood as to how these two processes are coordinated. One possible mechanism lies at the level of transcription where the expression of genes critical in both cellular energy production and utilization processes can be regulated in an orchestrated manner. However, such transcription coordinators that directly regulate multiple energy-generating cellular metabolic pathways and energy-consuming cellular functions remain to be identified.

The heart offers a system for studying coordination of energy production and consumption. It continuously pumps blood to all the organs, involving energy-demanding processes such as myocardial contraction and electrical conduction. Accordingly cardiomyocytes maintain an exceedingly high metabolic rate and depend on vigorous fatty acid oxidation (FAO), oxidative phosphorylation (OxPhos) and dynamic mitochondrial networks to generate energy that supports these functions. Indeed, defects in cardiomyocyte metabolism and mitochondrial function are underlying causes of or are associated with many cardiac diseases including cardiomyopathy and heart failure that affect millions of people.

Nuclear receptors (NRs) are ligand-activated transcription factors with roles in physiological and pathological settings. Among the 48 NRs in the human genome, several NRs and their coactivators have been identified as key regulators of cardiac metabolism. In particular, recent work has revealed roles for the estrogen-related receptor (ERR) subfamily of NRs especially ERRα and ERRγ in regulating cellular metabolism. Genomic studies have found that ERRα and ERRγ target a common set of promoters of genes related to FAO, OxPhos and muscle contraction (Dufour et al., 2007. Cell Metab 5:345-356). Whole body ERRα KO mouse hearts exhibit defects in the bioenergetic and functional adaptation to cardiac pressure overload, but their development and function under normal, unstressed conditions remain intact (Huss et al., 2007. Cell Metab 6:25-37). Whole body ERRγ KO mice display neonatal cardiac defects demonstrating the importance of ERRγ in supporting the transition to oxidative metabolism in the perinatal heart (Alaynick et al., 2007. Cell Metab 6:13-24). Unfortunately the neonatal lethality (100% within 48 hours) of the whole body ERRγ KO mice excluded further study of ERRγ. In addition, it is unclear whether these cardiac phenotypes are owing to cell autonomous functions of cardiomyocyte ERRγ. Furthermore, due to the potential overlapping target genes of ERRα and ERRγ, their in vivo physiological importance remains to be determined.

### SUMMARY

Disclosed herein is the finding that nuclear receptors ERRα and ERRγ are essential transcriptional coordinators of cardiac energy production and consumption. On the one hand, ERRα and ERRγ together are vital for intact cardiomyocyte metabolism by directly controlling expression of genes important for mitochondrial functions and dynamics. On the other hand, ERRα and ERRγ influence major cardiomyocyte energy-consumption functions through direct transcriptional regulation of key contraction, calcium homeostasis and conduction genes. Mice lacking both ERRα and cardiac ERRγ develop severe bradycardia, lethal cardiomyopathy and heart failure featuring metabolic, contractile and conduction dysfunctions. These results illustrate that the ERR transcriptional pathway is essential to couple cellular energy metabolism with energy consumption processes in order to maintain normal cardiac function.

Mice that specifically lack cardiac ERRγ, or both ERRα and cardiac ERRγ, were generated. While mice lacking either ERRα or cardiac ERRγ exhibited normal survival and cardiac functions, mice lacking both ERRα and cardiac ERRγ died within the first month of their life with evident cardiomyopathy and heart failure. Their hearts displayed multiple metabolic defects including mitochondrial fragmentation and significantly decreased OxPhos activity, accompanied with reduced expression of related genes which are ERR targets. Importantly, the dynamic mitochondrial networks were significantly disrupted, revealing an essential role for ERRα and ERRγ in controlling mitochondrial dynamics. It was further observed that this effect was mediated at least partially through direct transcriptional regulation of critical mitochondrial fusion proteins Mfn1 and Mfn2 by ERRα and ERRγ. It was also demonstrated that ERRα and ERRγ were required for integral cardiac contractile function via regulating genes important in contraction and calcium homeostasis. In addition, mouse hearts lacking ERRα and ERRγ exhibited severe bradycardia and abnormal electrocardiography (ECG), revealing their vital roles in myocardial conduction. Mechanistically, it is shown herein that ERRα and ERRγ directly bound to and regulated the transcription of many potassium, sodium and calcium channel genes implicated in human cardiac conduction disorders. Together these results demonstrate the fundamental roles of ERRα and ERRγ in coordinating the cellular energy production and consumption in the heart through orchestrated transcriptional regulation of both processes. These results demonstrate the therapeutic potential of targeting ERRα and ERRγ pathways for treating cardiac diseases, such as bradycardia, cardiomyopathy and heart failure.

Based on these observations, provided herein are methods of increasing cardiac contraction, increasing mitochondrial activity, and/or increasing oxphos activity, in a subject, such as a human or veterinary mammal. Such methods can include administering a (1) therapeutically effective amount of one or more agents that increases estrogen-related receptor (ERR) α activity and (2) a therapeutically effective amount of one or more agents that increases ERRγ activity, to a mammal needing increased cardiac contraction, increased mitochondrial activity, and/or increased oxphos activity. In some examples, the method further includes administering a therapeutically effective amount of one or more agents that increases mitofusin 1 (Mfn1) activity to the mammal needing increased cardiac contraction, increased mitochondrial activity, and/or increased oxphos activity.

Also provided are methods of increasing cardiac contraction, mitochondrial activity, and/or oxphos activity by at least 10%, wherein the increase of at least 10% as compared is relative to an amount of cardiac contraction, mitochondrial activity, and/or oxphos activity in the absence of administration of the one or more agents that increases ERRα activity and in the absence of administration of the one or more agents that increases ERRγ activity. Such methods can include administering a (1) therapeutically effective amount of one or more agents that increases estrogen-related receptor (ERR) α activity and (2) a therapeutically effective amount of one or more agents that increases ERRγ activity, to a mammal needing increased cardiac contraction, increased mitochondrial activity, and/or increased oxphos activity. In some examples, the method further includes administering a therapeutically effective amount of one or more agents that increases mitofusin 1 (Mfn1) activity to a mammal needing increased cardiac contraction, increased mitochondrial activity, and/or increased oxphos activity.

In some examples, the methods further include not exercising the mammal. In some examples, the methods further include selecting a mammal in need of increased cardiac contraction, increased mitochondrial activity, and/or increased oxphos activity or a mammal at risk for developing a disorder that can benefit from increased cardiac contraction, increased mitochondrial activity, and/or increased oxphos activity. Mammals that can be treated with the disclosed methods can include those having, or at risk for developing, heart failure, bradycardia and/or cardiomyopathy.

Also provided are kits that can be used with such methods. For example, such kits can include one or more agents that increases ERRα activity and one or more agents that increases ERRγ activity. In some examples, the kit also includes one or more agents that increases Mfn1 activity.

The foregoing and other objects and features of the disclosure will become more apparent from the following detailed description, which proceeds with reference to the accompanying figures.

### BRIEF DESCRIPTION OF THE DRAWINGS

FIGS. 1A-1D show that mice lacking cardiac ERRα and ERRγ die postnatally. (A) Myh6-Cre mediated cardiac specific loss of ERRγ. ERRγ, ERRα and ERRβ RNA in different tissues from 2 month old control (Cre−) and cardiac ERRγ KO (Cre+) mice (n=4-5) were determined by qRT-PCR. **p<0.01 between Cre- and Cre+. (B) ERRα and ERRγ RNA (top, n=4-8) and nuclear protein (bottom, n=2) level in 3 day old mouse hearts was determined by qRT-PCR and western blot, respectively. *p<0.05 and **p<0.01 between indicated genotype and αWTγWT mice. (C) Survival rate of pups at 0, 13 and 26 days of age (n=10-20). (D) Breeding strategy to generate experimental cohorts. All values are mean+s.e.m.

FIGS. 2A-2D show that mice lacking cardiac ERRα and ERRγ die postnatally with cardiomyopathy. (A) Heart weight of 16 day old mice (n=7-11). (B) Representative picture of hearts of 16 day old mice. (C) Representative pictures of H&E stained heart sections of 16 day old mice. Top—cross section of the heart; middle—longitudinal view of the muscle fibers; bottom—transverse view of the muscle fibers. (D) Expression of ANP and BNP in 16 day old mouse hearts (n=6-8). **p<0.01 between αKOγKO and all other 3 genotypes. All values are mean+s.e.m.

FIGS. 3A-3C show that ERRα and ERRγ are essential for expression of genes important in cardiomyocyte metabolism especially mitochondrial functions. Expression of genes important in mitochondrial (A) fatty acid oxidation, (B) biogenesis, and (C) OxPhos, in 16 day old mouse hearts (n=6-8). **p<0.01, ***p<0.001, ****p<0.0001, *****p<0.00001 and ******p<0.000001 between αKOγKO and all other 3 genotypes; ̂p<0.05 and ̂̂p<0.01 between αKOγWT and αHetγWT/αHetγKO. All values are mean+s.e.m.

FIGS. 4A-4C show that ERRα and ERRγ are essential for normal mitochondrial functions. (A) Representative EM images of hearts of 16 day old mice. Top row—magnification of 10,000× to show the overall cardiac myofibril structure including the sarcomeres and the mitochondria; middle row—magnification of 60,000× to show the mitochondria ultrastructure and the sarcomere Z line (Z), I band (I) and A band; bottom row—magnification of 60,000× to focus on the mitochondria ultrastructure. (B) Mitochondria size (2-dimensional area) and perimeter were quantified from 5 EM fields (magnification of 10,000×, at least 100 mitochondria per field) using Image J. (C) Activity of TCA cycle enzyme citrate synthase and different mitochondrial ETC complexes in 16 day old mouse hearts was measured by enzymatic assays (n=3). *p<0.05, **p<0.01 and ****p<0.0001 between αKOγKO and all other 3 genotypes; ̂̂̂̂p<0.0001 between αKOγWT and αHetγWT/αHetγKO. All values are mean+s.e.m.

FIGS. 5A-5F show ERRα and ERRγ are important for integral mitochondrial dynamics. (A) Representative EM pictures of 16 day old αKOγKO hearts. Magnification of 75,000× to show some mitochondria surrounded by multiple double membranes (*). (B) mtDNA/nDNA content in 16 day old mouse hearts (n=4). **p<0.01 between αKOγKO and all other 3 genotypes; ̂̂p<0.01 between αKOγWT and αHetγKO only. (C-D) Expression of Mfn1, Mfn2, Opa1 and Drp1 in 16 day (C) and 3 day (D) old mouse hearts (n=6-8). *p<0.05, **p<0.01 and *****p<0.00001 between αKOγKO and all other 3 genotypes; ̂̂̂̂p<0.0001 between αKOγWT and αHetγWT/αHetγKO. (E) ERRα and ERRγ bind to ERRγ within the first intron of the mouse Mfn1 gene and in the promoter region of the mouse Mfn2 gene. ChIP was performed in mouse HL-1 cardiomyocytes. **p<0.01 and ****p<0.0001 compared to IgG control. (F) ERRα and ERRγ directly activate the ERRE of the mouse Mfn1 and Mfn2 genes. Transient transfection was performed in 293 cells. The value was normalized to the pcDNA3.1/PGL4.10 group. *p<0.05 and **p<0.01 compared to pcDNA3.1 empty plasmid control. All values are mean+s.e.m.

FIGS. 6A-6C show that overexpression of Mfn1 partially rescues the mitochondrial morphology and mtDNA defects in αKOγKO MEFs. (A) Representative confocal microscopy images show the mitochondrial morphology (revealed via ATP5b protein staining) in WT (αWTγWT) and ERRα^{-/-}ERRγ^{-/-} (αKOγKO) MEFs infected with control mtDsRed or Mfn1 retrovirus. (B) Mitochondria (ATP5b positive staining) size (2-dimensional area) and perimeter in αWTγWT and αKOγKO MEFs were quantified (20-25 images per group). (C) mtDNA/nDNA content in αWTγWT and αKOγKO MEFs (n=3). *p<0.05, **p<0.01 and ****p<0.0001 between indicated genotype/treatment and the αKOγKO MEFs with mtDsRed (middle column). All values are mean+s.e.m.

FIGS. 7A-7D show that ERRα and ERRγ are vital for cardiac contractile function. (A) Representative echocardiography images (M-mode) of 15-16 day old mice (n=4). Part of the contraction track was marked by white lines for easy visualization. Since αKOγKO mice have lower heart rate (FIGS. 9A and B), the x-axis time scale of the αKOγKO echocardiography images were different from those of other genotypes. (B) LV mass and wall thickness in 15-16 day old mice measured by echocardiography. LVPWd—diastolic LV posterior wall thickness. (C) Cardiac dimensions and volumes of 15-16 day old mice (n=4) measured by echocardiography. LVIDd—left ventricle internal dimension, diastolic; LV EDV—left ventricle volume, end of diastolic; LVIDs left ventricle internal dimension, systolic; LV ESV—left ventricle volume, end of systolic. (D) Ejection fraction (EF) and cardiac output (CO) in 15-16 day old mice (n=4) measured by echocardiography. *p<0.05 and ***p<0.001 between αKOγKO and all other 3 genotypes. Values are mean±s.e.m.

FIGS. 8A-8B show ERRα and ERRγ regulate cardiac contraction through transcriptional modulation of muscle contractile genes. (A) Expression of genes important in cardiac contraction in 16 day old mouse hearts (n=6-8). *p<0.05, **p<0.01, ***p<0.001 and ****p<0.0001 between αKOγKO and all other 3 genotypes; ̂p<0.05 between αKOγWT and αHetγWT/αHetγKO; ^{#}p<0.05 between αKOγWT/αHetγKO and αHetγWT. (B) ERRα and ERRγ bound to ERRE within the first introns of the mouse Tnnc1 and Tnnt2 genes. ChIP was performed in HL-1 cardiomyocytes. **p<0.01 and ****p<0.0001 compared to IgG control. All the values are mean+s.e.m.

FIGS. 9A-9F show that ERRα and ERRγ are essential for normal myocardial conduction through transcriptional regulation of key potassium, sodium and calcium channels. (A) Representative ECG of 16 day old mice. (B) Heart rate, PR interval, QRS complex and QT interval in 16 day old mice (n=7-9) measured by ECG. **p<0.01 and *****p<0.00001 between αKOγKO and all other 3 genotypes. (C) Expression of ion channel genes implicated in human conduction disorders in 16 day old mouse hearts (n=6-8). *p<0.05, **p<0.01, ***p<0.001 and ****p<0.0001 between αKOγKO and all other 3 genotypes. (D) ERRα and ERRγ bound to ERRE within the first intron of the mouse Kcnq1 and Kcnh2 genes. ChIP was performed in HL-1 cardiomyocytes. **p<0.01 and ****p<0.0001 compared to IgG control. (E) ERRα and ERRγ directly activate the ERRE of the mouse Kcnq1 and Kcnh2 genes. Transient transfection was performed in 293 cells. The value was normalized to the pcDNA3.1/PGL4.10 group. *p<0.05 and **p<0.01 compared to pcDNA3.1 empty plasmid control. In (B-E) all values are mean+s.e.m. (F) Global potassium currents recorded from single ventricular myocyte isolated from 12-16 day old hearts (n=5). Shown on the top are representative single cell potassium currents recorded from each of the four different genotypes. The plot on the bottom shows the normalized peak current density versus membrane potential, where the insert depicts the pulse protocol applied to elicit the family of currents. Values are mean±s.e.m. *p<0.05 between αKOγKO and all other 3 genotypes.

### SEQUENCE LISTING

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing generated on Sep. 17, 2018, 63.4 Kb, is herein incorporated by reference.

SEQ ID NOS: 1-12 are primer sequences used for ChIP analysis.

SEQ ID NOS: 13-20 are primer sequences used for mtDNa/nDNA analysis.

SEQ ID NOS: 21-94 are primer sequences used for qRT-PCR analysis.

SEQ ID NOS: 95 and 96 are exemplary ERRα coding and amino acid sequences, respectively.

SEQ ID NOS: 97 and 98 are exemplary ERRγ coding and amino acid sequences, respectively.

SEQ ID NOS: 99 and 100 are exemplary Mfn1 coding and amino acid sequences, respectively.

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All publications, patent applications, patents, and other references mentioned herein are incorporated by reference in their entirety for all purposes. All sequences associated with the GenBank® Accession numbers mentioned herein are incorporated by reference in their entirety as were present on Apr. 2, 2015. Although exemplary GenBank® numbers are listed herein, the disclosure is not limited to the use of these sequences. Many other ERRα, ERRγ and Mfn1 sequences are publicly available, and can thus be readily used in the disclosed methods. In one example, an ERRα, ERRγ, or Mfn1 sequence has at least 70%, at least 75%, at least 80%, at least 85%, at least 90%, at least 95%, at least 98%, or at least 100% sequence identity to any of the GenBank® numbers are listed herein.

In order to facilitate review of the various embodiments of the disclosure, the following explanations of specific terms are provided:

Administration: The introduction of a composition, such as an ERRα and/or ERRγ agonist, into a subject by a chosen route, for example topically, orally, intravascularly such as intravenously, intramuscularly, intraperitoneally, intranasally, intradermally, transdermally, intrathecally, subcutaneously, via inhalation or via suppository. Administration can be local or systemic, such as intravenous or intramuscular. For example, if the chosen route is intravenous, the composition is administered by introducing the composition into a vein of the subject. In some examples an ERRα agonist, ERRγ agonist, and/or Mfn1 agonist is administered to a subject at an effective dose.

Estrogen receptor-related receptor α (ERR α): (e.g., OMIM 601998) An orphan nuclear receptor of the ERR subfamily. This protein is related to the estrogen receptor, and may be required for the activation of mitochondrial genes. This protein acts as a site-specific (consensus TNAAGGTCA) transcription regulator and has been also shown to interact with estrogen and the transcription factor TFIIB by direct protein-protein contact.

ERRα sequences are publicly available. For example, GenBank® Accession Nos. NM_001282450.1 and NM_007953 disclose ERRα nucleic acids, and GenBank® Accession Nos. NP_001269379 and NP_031979 disclose ERRα proteins. In certain examples, ERRα has at least 80% sequence identity, for example at least 85%, 90%, 95%, or 98% sequence identity to such sequences (such as SEQ ID NOS: 95 and 96), and retains ERRα activity.

ERRα activity includes the ability to increase cardiac contraction, increase mitochondrial activity (such as mitochondrial respiration), and/or increase oxphos activity (for example in cardiac muscle).

Estrogen receptor-related receptor γ (ERRγ): (e.g., OMIM 602969) A constitutively active orphan nuclear receptor of the ERR subfamily. Unlike ERRα and β, it is more selectively expressed in metabolically active and highly vascularized tissues such as heart, kidney, brain and skeletal muscles.

ERRγ sequences are publicly available. For example, GenBank® Accession Nos. NM_001134285.2, AY388461, AF058291.1 and NM_011935.2 disclose ERRγ nucleic acids, and GenBank® Accession Nos. NP_001127757.1, P62508.1, AAQ93381.1, and NP_036065.1 disclose ERRγ proteins. In certain examples, ERRγ has at least 80% sequence identity, for example at least 85%, 90%, 95%, or 98% sequence identity to such sequences (such as SEQ ID NOS: 97 and 98), and retains ERRγ activity.

ERRγ activity includes the ability to increase cardiac contraction, increase mitochondrial activity (such as mitochondrial respiration), and/or increase oxphos activity (for example in cardiac muscle).

Isolated: An “isolated” biological component (such as a nucleic acid, protein or antibody) has been substantially separated, produced apart from, or purified away from other biological components in the cell of the organism in which the component naturally occurs, such as, other chromosomal and extrachromosomal DNA and RNA, and proteins. Nucleic acids and proteins which have been “isolated” thus include nucleic acids and proteins purified by standard purification methods. The term also embraces nucleic acids and proteins prepared by recombinant expression in a host cell as well as those chemically synthesized.

Mitofusin-1 (Mfn1): (e.g., OMIM 608506) A mediator of mitochondrial fusion.

Mfn1 sequences are publicly available. For example, GenBank® Accession Nos. NM_033540.2, NM_024200.4, NM_001206508.1 and NM_138976.1 disclose Mfn1 nucleic acids, and GenBank® Accession Nos. Q8IWA4.2, NP_077162.2, NP_001193437.1, and NP_620432.1 disclose Mfn1 proteins. In certain examples, Mfn1 has at least 80% sequence identity, for example at least 85%, 90%, 95%, or 98% sequence identity to such sequences (such as SEQ ID NOS: 99 and 100), and retains Mfn1 activity.

Pharmaceutically acceptable carriers: The pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of an ERRα agonist, ERRγ agonist, and/or Mfn1 agonist or other agent that increases ERRα, ERRγ and/or Mfn1 activity.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Recombinant: A recombinant nucleic acid molecule or protein is one that has a sequence that is not naturally occurring or has a sequence that is made by an artificial combination of two otherwise separated segments of sequence. This artificial combination can be accomplished by methods known in the art, such as chemical synthesis or by the artificial manipulation of isolated segments of nucleic acids, e.g., by genetic engineering techniques. Cells that express such molecules are referred to as recombinant or transgenic cells.

Sequence identity: The similarity between amino acid or nucleic acid sequences are expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of ERRγ that retain ERRγ activity are encompassed by this disclosure typically characterized by possession of at least about 75%, for example at least about 80%, at least 85%, at least 90%, at least 91%, at least 92%, at least 93%, at least 94%, at least 95%, at least 96%, at least 97%, at least 98% or at least 99% sequence identity counted over the full length alignment with the amino acid or nucleic acid sequence of interest, such as SEQ ID NO: 97 or 98. Similarly, variants of ERRα that retain ERRα activity are encompassed by this disclosure typically characterized by possession of at least about 75%, for example at least about 80%, at least 85%, at least 90%, at least 91%, at least 92%, at least 93%, at least 94%, at least 95%, at least 96%, at least 97%, at least 98% or at least 99% sequence identity counted over the full length alignment with the amino acid or nucleic acid sequence of interest, such as SEQ ID NO: 95 or 96. Variants of Mfn1 that retain Mfn1 activity are encompassed by this disclosure typically characterized by possession of at least about 75%, for example at least about 80%, at least 85%, at least 90%, at least 91%, at least 92%, at least 93%, at least 94%, at least 95%, at least 96%, at least 97%, at least 98% or at least 99% sequence identity counted over the full length alignment with the amino acid or nucleic acid sequence of interest, such as SEQ ID NO: 99 or 100. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes human and non-human mammals, such as veterinary subjects (e.g., cats, dogs, horses, cows, pigs, goats, mice and rats).

Therapeutically effective amount: An amount of a pharmaceutical preparation that alone, or together with a pharmaceutically acceptable carrier or one or more additional therapeutic agents, induces the desired response. A therapeutic agent, such as an ERRα agonist, ERRγ agonist, and/or Mfn1 agonist, is administered in therapeutically effective amounts. In some embodiments, a therapeutically effective amount is the amount of one or more agents that increase ERRα, ERRγ, and/or Mfn1 activity necessary to increase or more of cardiac contraction, mitochondrial activity (such as mitochondrial respiration), and/or increase oxphos activity (for example in cardiac muscle), such as increases of at least 20%, at least 50%, at least 60%, at least 75%, at least 80%, or at least 95% as compared to an absence of the one or more agents that increase ERRα, ERRγ, and/or Mfn1activity. When administered to a subject, a dosage will generally be used that will achieve target tissue concentrations that has been shown to achieve a desired in vitro effect.

Effective amounts a therapeutic agent can be determined in many different ways, such as assaying for an increase in cardiac contraction, mitochondrial activity e, and/or oxphos activity, or improvement of physiological condition of a subject having or at risk for a disease such as a mitochondrial disease or cardiac disease (such as heart failure or cardiomyopathy). Effective amounts also can be determined through various in vitro, in vivo or in situ assays.

Therapeutic agents can be administered in a single dose, or in several doses, for example daily, during a course of treatment. However, the effective amount of can be dependent on the source applied, the subject being treated, the severity and type of the condition being treated, and the manner of administration.

Treating a disease: “Treatment” refers to a therapeutic intervention that ameliorates a sign or symptom of a disease or pathological condition after it has begun to develop, such a sign or symptom of a mitochondrial disease or cardiac disease (such as bradycardia, heart failure or cardiomyopathy). Treatment can also induce remission or cure of a condition, such as heart failure or cardiomyopathy. Preventing a disease refers to a therapeutic intervention to a subject who does not exhibit signs of a disease or exhibits only early signs for the purpose of decreasing the risk of developing pathology, such that the therapy inhibits or delays the full development of a disease, such as preventing development of a mitochondrial disease or cardiac disease (such as heart failure, bradycardia, or cardiomyopathy). Treatment and prevention of a disease does not require a total absence of disease. For example, a decrease of at least 20% or at least 50% can be sufficient. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

Upregulated or activation: When used in reference to the expression of a nucleic acid molecule, such as an ERRα, ERRγ, and/or Mfn1 gene, refers to any process which results in an increase in production of a gene product. A gene product can be RNA (such as mRNA, rRNA, tRNA, and structural RNA) or protein (such as an ERRα, ERRγ, and/or Mfn1 protein). Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 2-fold, for example at least 3-fold or at least 4-fold, as compared to a control (such an amount of gene expression in an untreated cell, such as a cell not contacted with an agent that increases ERRα, ERRγ, and/or Mfn1 activity).

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

### Overview

Whole body ERRγ KO pups die shortly after birth. This neonatal lethality (100% penetrance within 48 hours) happens at both the originally reported ICR outbred and C57BL6/J inbred backgrounds. The neonatal ERRγ KO pups show altered expression of some cardiac metabolic and contractile genes but little functional consequences, and they maintain normal cardiac structure. At the time of those studies, it was not clear whether the neonatal lethality was caused by these cardiac problems or other unidentified defects in other tissues. The demonstration herein that the normal survival and cardiac function of cardiac-specific ERRγ KO mice indicates that non-cardiac actions of ERRγ are critical in supporting the neonatal survival in mice. In addition to the heart, ERRγ is also abundantly expressed in other tissues including the brain and kidney. Whole body ERRγ KO mouse also displayed defects in embryonic kidney development (Berry et al., 2011. Hum Mol Genet 20:917-926).

Prior in vitro studies indicated that ERRα and ERRγ are regulators of cardiac metabolism. However, neither ERRα KO nor cardiac-specific ERRγ KO mice exhibited any major cardiac structural or functional defects at basal states. The data herein firmly establish the essential roles of ERRα and ERRγ together in maintaining intact cardiac metabolism and function. In addition to providing definitive evidence supporting the importance of ERRα and ERRγ in regulating cellular oxidative metabolism and cardiac contractile function, it is shown herein that ERRα and ERRγ are essential for other aspects of cardiac physiology. First, they regulate mitochondrial dynamics through direct transcriptional regulation of key mitochondrial fusion genes. αKOγKO hearts exhibit loss of mtDNA and defective mitochondrial dynamics with concomitant decreased expression of key mitochondrial dynamics genes such as Will. These defects can be partially rescued by restored expression of Mfn1. Second, ERRα and ERRγ directly control the expression of many ion channel genes essential for intact myocardial conduction. Although the possible role of ERRα in regulating the expression of some of these genes was implicated from gain-of-function studies in different cell types (Tremblay et al., 2010. Mol Endocrinol 24:22-32; Liesa et al., 2008. PLoS One 3:e3613; Cartoni et al., 2005. J Physiol 567:349-358; Soriano et al., 2006. Diabetes 55:1783-1791.), the results herein provide the first definitive evidence that ERRα and ERRγ together are required for their expression in a loss-of-function context and are absolutely essential for integral mitochondrial dynamics and myocardial conduction in vivo. Since cardiac bioenergetic deficiency, contractile dysfunction or conduction defect alone can result in cardiomyopathy and associated cardiac dysfunctions, it is likely that such indirect effects and ERR-dependent direct transcriptional regulation both contribute to the overall cardiomyopathy phenotype of αKOγKO mice.

The phenotype of the αKOγKO mice provided herein are strikingly similar to those reported in whole body Pgc1α/Pgc1βKO (or cardiac Pgc1βKO in the whole body Pgc1α KO background) or cardiac Mfn1/Mfn2 KO mice (Lai et al., 2008. Genes Dev 22:1948-1961.). These include early-onset, 100% penetrant postnatal lethality, cardiomyopathy, bradycardia and cardiac dysfunction together with defects in cellular metabolism, mitochondrial structure and function. The Mfn1 locus impacts heart rate in humans through GWAS studies, and its knockdown reduced heart rate in both fruit fly and zebrafish (den Hoed M et al., 2013. Nat Genet 45:621-631). These raise the possibility that these cardiac phenotypes are controlled by a common cellular pathway involving ERR, Pgc1 and Mfn proteins. Pgc1α and Pgc1β are coactivators of many transcription factors including ERRα and ERRγ, playing important roles in many physiological and pathological conditions (Lin et al., 2005. Cell Metab 1:361-370). The phenotypic similarity between Pgc1α/Pgc1βKO and the disclosed cardiac ERRα/ERRγ KO mice suggest that ERRα and ERRγ are the principal transcription factor partners of the Pgc1 proteins in the developing mouse heart. In addition, Mfn1 and Mfn2 are downstream targets of both ERRα/ERRγ and Pgc1α/Pgc1β signaling. Thus, ERRα/ERRγ, together with coactivators Pgc1α/Pgc1β, may control cardiac metabolism and function at least partially through Mfn1/Mfn2.

Cellular energy production and consumption are coordinated to support various cellular functions. Through regulating multiple processes involved in cellular energy production (FAO, OxPhos and mitochondrial dynamics) and consumption (cardiac contraction, calcium homeostasis and electrical conduction) at the same time, ERRα and ERRγ offer the critical assistance to this supply-demand relationship. Mechanistic studies demonstrate that a large number of genes critical in all these pathways are direct transcriptional targets of ERRα and ERRγ. In addition, ERRα transcriptional activity can be modulated by multiple signaling pathways that reflect either cellular physiological/energy status or metabolic demand from distinct cellular functions.

Based on the results provided herein, methods of using agents that alter expression or activity of ERRα and/or ERRγ to treat heart diseases such as cardiomyopathy and heart failure are provided. Altered expression of ERRα and ERRγ as well as mutations of their target genes have been associated with cardiomyopathy, heart failure and conduction disorders in humans (Gupte et al., 2014. Circ Cardiovasc Genet. 7:266-76; Hu et al., 2011. Hypertension 58:696-703; Kwon et al., 2013. J Mol Cell Cardiol 65:88-97). Like many other nuclear receptors, the activities of ERRα and ERRγ can be modulated by available small-molecule ligands (C29, XCT790, GSK5182, GSK4716, etc.) (Kwon et al., 2013. J Mol Cell Cardiol 65:88-97; Kim et al., 2014. Nat Med 20:419-424; Patch et al., 2011. J Med Chem 54:788-808; Chaveroux et al., 2013. Cell Metab 17:586-598).

### Methods of Treatment

Provided herein are methods of increasing cardiac contraction, increasing mitochondrial activity, increasing oxphos activity, or combinations of these (such as 1, 2 or all 3 of these). Such methods can include administering a therapeutically effective amount of one or more agents that increases estrogen-related receptor (ERR) α activity and administering a therapeutically effective amount of one or more agents that increases ERRγ activity, to a mammal needing increased cardiac contraction, increased mitochondrial activity, and/or increased oxphos activity. Also provided are methods of increasing cardiac contraction, mitochondrial activity, and/or oxphos activity by at least 10%. Such methods can include administering a therapeutically effective amount of one or more agents that increases ERRα activity to a mammal needing increased cardiac contraction, increased mitochondrial activity, and/or increased oxphos activity and administering a therapeutically effective amount of one or more agents that increases ERRγ activity to a mammal needing increased cardiac contraction, increased mitochondrial activity, and/or increased oxphos activity; wherein the increase of at least 10% as compared is relative to an amount of cardiac contraction, mitochondrial activity, and/or oxphos activity in the absence of administration of the one or more agents that increases ERRα activity and in the absence of administration of the one or more agents that increases ERRγ activity. In some examples, such methods are used to treat or prevent heart failure, bradycardia and/or cardiomyopathy.

In some examples, the methods further include administering a therapeutically effective amount of one or more agents that increases mitofusin 1 (Mfn1) activity (such as a nucleic acid molecule encoding Mfn1, one or more Mfn1 agonists, an Mfn1 protein; or combinations thereof) to a mammal needing increased cardiac contraction, increased mitochondrial activity, and/or increased oxphos activity. In some examples, the method further includes not exercising the mammal being treated with the disclosed methods. In some examples, the method further includes selecting a mammal in need of increased cardiac contraction, increased mitochondrial activity, and/or increased oxphos activity or a mammal at risk for developing a disorder that can benefit from increased cardiac contraction, increased mitochondrial activity, and/or increased oxphos activity.

Thus, in some examples, the treated mammal has or is at risk for heart failure, bradycardia and/or cardiomyopathy. In some examples, the mammal cannot exercise or is sedentary. Examples of subjects that can be treated with the disclosed therapies include humans and veterinary subjects, such as dogs, cats, mice, and rats. Any mode of administration can be used, such as parenteral, subcutaneous, intraperitoneal, intrapulmonary, or intranasal administration.

The disclosed methods can be used in combination with other therapies, such as other therapies used to treat heart failure, bradycardia and/or cardiomyopathy. Thus, in some examples, the disclosed methods further include administration of a therapeutically effective amount of an agent that balances electrolytes (e.g., aldosterone antagonists such as spironolactone or eplerenone), an agent that prevents arrhythmias (antiarrhythmics such as amiodarone, bepridil hydrochloride, disopyramide, dofetilide, dronedarone, flecaninide, ibutilide, lidocaine, procainamide, propafenone, propranolol, quinidine, sotalol, and tocanide); an agent that lowers blood pressure (e.g., ACE inhibitors [such as benazepril, captopril, enalapril, fosinopril, lisinopril, moexipril, perindopril, quinapril, ramipril, and trandolapril]; angiotensin II receptor blockers [such as losartan, candesartan, valsartan, irbesartan, telmisartan, eprosatan, olmesartan, azilsartan, and fimasartan), beta blockers [such as acebutolol, atenolol, betaxolol, bisoprolol, celiprolol, esmolol, metoprolol, landiolol, labetalol, carvedilol, and nebivolol], and calcium channel blockers [such as dihydropyridines (e.g., amlodipine, barnidipine, efonidipine) and non-dihydropyridines (e.g., phenylalkylamines such as verapamil, gallopamil, and fendiline; and benzothiazepines such as ditiazem)], an agent that prevents blood clots from forming (e.g., anticoagulants such as dabigatran, rivaroxaban, apixaban, edoxban, coumarin, acenocoumarol, phenprocoumon, atromentin, phenindione, heparin, rivaroxaban, apixaban edoxaban, acenocoumarol, phenprocoumon, atromentin, and phenindione), an agent that reduces inflammation (e.g., corticosteroids such as corticosterone, cortisone, aldosterone), an agent that removes excess sodium (e.g., diuretics such as thiazides (e.g., hydrochlorothiazide), acetazolamide, methazolamide, lasix, bumex, aldactone, dyrenium, and mannitol), an agent that decreases heart rate (e.g., beta blockers [such as acebutolol, atenolol, betaxolol, bisoprolol, celiprolol, esmolol, metoprolol, landiolol, labetalol, carvedilol, and nebivolol], calcium channel blockers [such as dihydropyridines (e.g., amlodipine, barnidipine, efonidipine) and non-dihydropyridines (e.g., phenylalkylamines such as verapamil, gallopamil, and fendiline; and benzothiazepines such as ditiazem)], and digoxin), isosorbide dinitrate/hydralazine hydrochloride, or combinations thereof (such as 1, 2, 3, 4 or 5 of such agents). Appropriate dosages and modes of administration are known in the art.

In some examples, the method increases cardiac contraction by at least 10%, at least 20%, at least 30%, at least 40%, at least 50%, or at least 100%, for example as compared to the cardiac contraction in the absence of the disclosed therapy (e.g., before therapy is started). In some examples, the method increases mitochondrial activity (e.g., in the heart muscle) by at least 10%, at least 20%, at least 30%, at least 40%, at least 50%, or at least 100%, for example as compared to the mitochondrial activity (e.g., in the heart muscle) in the absence of the disclosed therapy (e.g., before therapy is started). In some examples, the method increases oxphos activity (e.g., in the heart muscle) by at least 10%, at least 20%, at least 30%, at least 40%, at least 50%, or at least 100%, for example as compared to the oxphos activity (e.g., in the heart muscle) in the absence of the disclosed therapy (e.g., before therapy is started).

Also provided are kits that can be used with such methods. For example, such kits can include one or more agents that increases ERRα activity and one or more agents that increases ERRγ activity. In some examples, the kit also includes one or more agents that increases Mfn1 activity. In some examples, the kit also includes one or more agents used to treat heart failure, bradycardia and/or cardiomyopathy, such as one or more agents listed above. In some examples, the reagents of the kit are in separate vials.

The one or more agents that increases ERRγ activity used in the method or kit can be a nucleic acid molecule encoding ERRγ, one or more ERRγ agonists, an ERRγ protein, or combinations thereof. In one example, the one or more agents that increases ERRγ activity is:

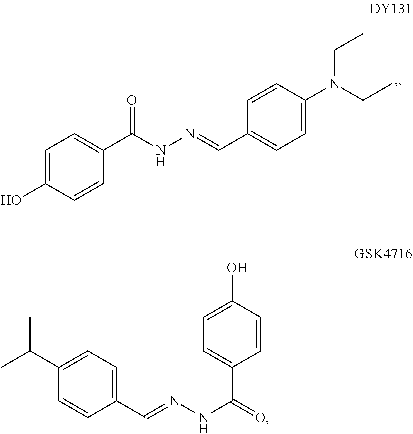

or combinations thereof. In another example, the one or more agents that increases ERRγ activity is:

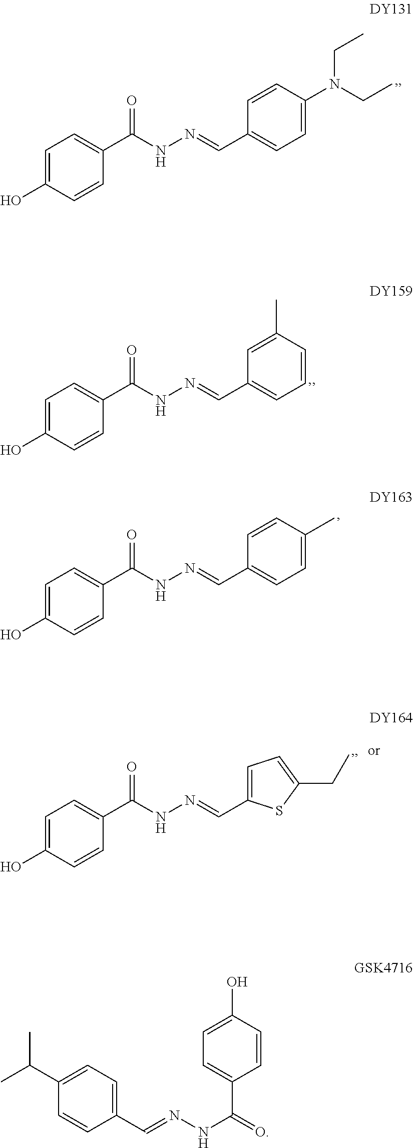

In one example, the one or more agents that increases ERRγ activity is:

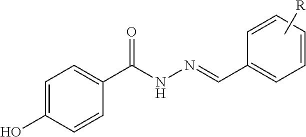

wherein R is H (DY162), p-CH₃ (DY163), 2-Cl, 3-CF₃ (DY165), p-CF₃ (DY168), p-OCH₃ (DY169), 3-NO₂, 4CF₃ (DY170), 2,3-O₂CH₃(DY174), or m-CH₃ (DY159),

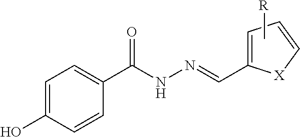

wherein X is S and R is 5-CH₃ (DY166), 5-CH₂CH₃ (DY164), or 5-NO₂ (DY167); wherein X is O and R is 4,5-CH₃ (DY173) or CH₂CH₃ (DY175), or wherein X is CH, and R is 2-Cl, 3-CF₃, p-CF₃; p-OCH₃, 3-NO₂, 4-CF₃; or 2,3-O₂CH₃;

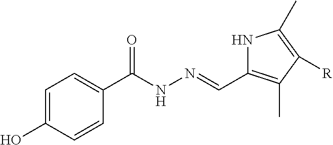

wherein R is H (DY117) or R is Br (DY172),

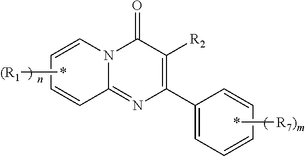

wherein
m is 0, 1 or 2;
n is 0, 1 or 2;
R₁ and R₇ are independently selected from
   1) H;
   2) Halo;
   3) OH;
   4) (C═O)ₐ, O_{b}C₁-C₄ alkyl, wherein a is 0 or 1 and b is 0 or 1, wherein the alkyl can be substituted by 0, 1 or more substituted groups independently selected from H or C₃C₆ heterocyclyl;
   5) (C═O), O_{b}C₃-C₆ cycloalkyl, wherein a is 0 or 1 and b is 0 or 1;
R2 is selected from:
   1) H;
   2) C₁-C₃ alkyl, wherein the alkyl can be substituted by 0, 1 or more substituted groups independently selected from H or C₃C₆ heterocyclyl;
   3) C₃-C₆ cycloalkyl;

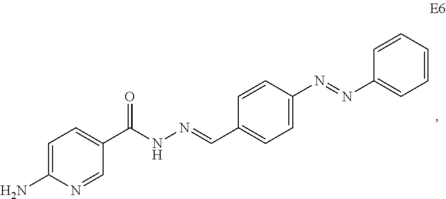 [SMILES: Nc1ccc(C(=O)N/N=C/c2ccc(/N=N/c3ccccc3)cc2)cn1]

or combinations thereof.

The one or more agents that increases ERRα activity used in the method or kit can be a nucleic acid molecule encoding ERRα, one or more ERRα agonists, an ERRα protein, or combinations thereof. In one example, the one or more agents that increases ERRα activity is:

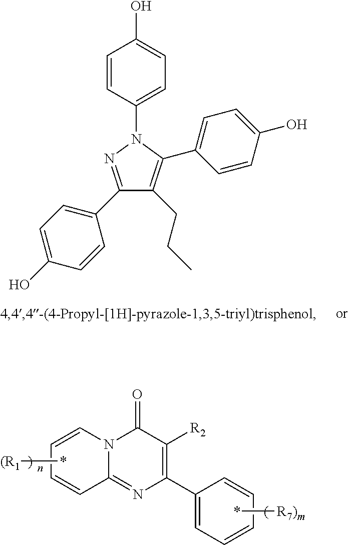

wherein:
m is 0, 1 or 2;
n is 1 or 2;
in the following, a is 0 or 1, b is 0 or 1;
each occurrence of R₁ is independently selected from the group consisting of:
   1) Halo;
   2) OH;
   3) (C══O)ₐO_{b}C₁-C₄ alkyl; and
   4) (C══O)ₐO_{b}C₃-C₆ cycloalkyl,
   wherein one occurrence of R₁ is at the 8-position of the pyrido [1,2-a]pyrimidin-4-one ring;
each occurrence of R₂ is independently selected from the group consisting of:
   1) Halo;
   2) OH;
   3) (C══O)ₐO_{b}C₁-C₄ alkyl, wherein, if a is 0 and b is 1, the alkyl is substituted with C₃-C₆ heterocyclyl; or
   4) (C══O)ₐO_{b}C₃-C₆ cycloalkyl;
   wherein, if one occurrence R₇ is halo of C₁-C₄ alkyl, at least one occurrence of R₁ is OH or (C══O)ₐO_{b}C₁-C₄ alkyl wherein a is 0 and b is 1;
      R₂ is selected from the group consisting of:
1) H;
2) C₁-C₃ alkyl; and
3) C₃-C₆ cycloalkyl;
the alkyl mentioned above can be substituted by 0, 1 or more substituted R₄ groups wherein R₄ is selected from the group consisting of:
   1) H; and
   2) C₃-C₆ heterocyclyl,
   or combinations thereof.

### Increasing Biological Activity

The present disclosure provides methods and pharmaceutical compositions for increasing cardiac contraction, increasing mitochondrial activity, and/or increasing oxphos activity, for example to treat or prevent a cardiac disorder, such as cardiomyopathy or heart failure.

ERRγ activity may be increased by increasing the amount of ERRγ protein being produced or by enhancing the activity of ERRγ protein. This can be achieved, for example, by administering a nucleotide sequence encoding for an ERRγ protein, an agent which enhances ERRγ expression, a substantially purified ERRγ protein, or an ERRγ agonist. An ERRγ agonist includes compounds which increase the ERRγ activity in a cell or tissue.

ERRα activity may be increased by increasing the amount of ERRα protein being produced or by enhancing the activity of ERRα protein. This can be achieved, for example, by administering a nucleotide sequence encoding for an ERRα protein, an agent which enhances ERRα expression, a substantially purified ERRα protein, or an ERRα agonist. An ERRα agonist includes compounds which increase the ERRα activity in a cell or tissue.

Mfn1 activity may be increased by increasing the amount of Mfn1 protein being produced or by enhancing the activity of Mfn1 protein. This can be achieved, for example, by administering a nucleotide sequence encoding for an Mfn1 protein, an agent which enhances Mfn1 expression, a substantially purified Mfn1 protein, or an Mfn1 agonist. An Mfn1 agonist includes compounds which increase the Mfn1 activity in a cell or tissue.

The particular mode of administration and the dosage regimen of the one or more agents that increase ERRα activity, ERRγ activity, and Mfn1 activity (or other therapeutic agent that can a treat or prevent a cardiac disorder, such as cardiomyopathy or heart failure) can be selected by the attending clinician, taking into account the particulars of the case (e.g. the subject, the disease, the disease state involved, the particular treatment, and whether the treatment is prophylactic). Treatment can involve daily or multi-daily (e.g., 2, 3, or 4 times daily) or less than daily (such as weekly or monthly etc.) doses over a period of a few days to months, or even years. In some examples, a therapeutic protein (such as a protein having at least 90%, at least 92%, at least 95%, at least 96%, at least 97%, at least 98%, at least 99% or 100% sequence identity to SEQ ID NO: 96, 98 or 100) is administered at a dose of at least 0.01 mg protein/kg, at least 0.1 mg/kg, or at least 0.5 mg/kg, such as about 0.01 mg/kg to about 50 mg/kg, for example, about 0.5 mg/kg to about 25 mg/kg, about 1 mg/kg to about 5 mg/kg, or about 1 mg/kg to about 10 mg/kg, for example about 2 mg/kg. In some examples, a therapeutic protein (such as a protein having at least 90%, at least 92%, at least 95%, at least 96%, at least 97%, at least 98%, at least 99% or 100% sequence identity to SEQ ID NO: 96, 98 or 100) is administered at a dose of at least 0.01 mg, at least 0.1 mg, at least 0.5 mg, at least 1 mg, or at least 1 g of protein, such as about 10 mg to about 100 mg, about 50 mg to about 500 mg, about 100 mg to about 900 mg, about 250 mg to about 750 mg, or about 400 mg to about 600 mg of protein. In some examples, a therapeutic nucleic acid (such as a nucleic acid having at least 90%, at least 92%, at least 95%, at least 96%, at least 97%, at least 98%, at least 99% or 100% sequence identity to SEQ ID NO: 95, 97 or 99, which may be part of a vector) is administered at a dose of at least 0.01 mg/kg, at least 0.1 mg/kg, or at least 0.5 mg/kg, such as about 0.01 mg/kg to about 50 mg/kg, for example, about 0.5 mg/kg to about 25 mg/kg, about 1 mg/kg to about 5 mg/kg, or about 1 mg/kg to about 10 mg/kg, for example about 2 or 3 mg/kg. In some examples, a therapeutic nucleic acid (such as a nucleic acid having at least 90%, at least 92%, at least 95%, at least 96%, at least 97%, at least 98%, at least 99% or 100% sequence identity to SEQ ID NO: 95, 97 or 99, which may be part of a vector) is administered at a dose of at least 0.00001 mg, at least 0.0001 mg, at least 0.001 mg, at least 0.01 mg, or at least 0.5 mg of nucleic acid, such as about 0.1 tag to about 100 μg, about 1 ng to about 500 ng, about 10 ng to about 10 μg, or about 100 ng to about 1 mg of nucleic acid. In some examples, a therapeutic agonist is administered at a dose of 0.01 mg agonist/kg to about 50 mg/kg, for example, about 0.5 mg/kg to about 25 mg/kg, about 1 mg/kg to about 5 mg/kg, or about 1 mg/kg to about 10 mg/kg. In some examples, a therapeutic agonist is administered at a dose of at least 0.01 mg, at least 0.1 mg, at least 0.5 mg, at least 1 mg, or at least 1 g of agonist, such as about 10 mg to about 100 mg, about 50 mg to about 500 mg, about 100 mg to about 900 mg, about 250 mg to about 750 mg, or about 400 mg to about 600 mg of agonist.

Administration of Proteins

In one example, ERRγ activity is increased by administering to the subject an ERRγ protein, such as a pharmaceutical composition containing such a protein. ERRγ protein sequences are known. For example, GenBank® Accession Nos. NP_001127757.1, P62508.1, AAQ93381.1, and NP_036065.1 disclose exemplary ERRγ protein sequences. However, one skilled in the art will appreciate that variations of such proteins can also retain ERRγ activity. For example such variants may include one or more amino acid deletions, substitutions, or additions (or combinations thereof), such as 1-50 of such changes (such as 1-40, 1-30, 1-20, or 1-10 of such changes). In certain examples, ERRγ has at least 80%, at least 85%, at least 90%, at least 95%, at least 97%, at least 98% or at least 99% sequence identity to such sequences (such as SEQ ID NO: 98), and retains ERRγ activity. In some examples, changes are not made to the ERRγ ligand binding domain (LBD). In some examples, residues Asp328, Arg316 and/or Asp275 are not changed.

In one example, ERRα activity is increased by administering to the subject an ERRα protein, such as a pharmaceutical composition containing such a protein. ERRα protein sequences are known. For example, GenBank® Accession Nos. NP_001269379 and NP_031979 disclose exemplary ERRα protein sequences. However, one skilled in the art will appreciate that variations of such proteins can also retain ERRα activity. For example such variants may include one or more amino acid deletions, substitutions, or additions (or combinations thereof), such as 1-50 of such changes (such as 1-40, 1-30, 1-20, or 1-10 of such changes). In certain examples, ERRα has at least 80%, at least 85%, at least 90%, at least 95%, at least 97%, at least 98% or at least 99% sequence identity to such sequences (such as SEQ ID NO: 96), and retains ERRα activity. In some examples, changes are not made to the ERRα ligand binding domain (LBD).

In one example, Mfn1 activity is increased by administering to the subject an Mfn1 protein, such as a pharmaceutical composition containing such a protein. ERRα protein sequences are known. For example, GenBank® Accession Nos. Q8IWA4.2, NP_077162.2, NP_001193437.1, and NP_620432.1 disclose exemplary Mfn1 protein sequences. However, one skilled in the art will appreciate that variations of such proteins can also retain ERRα activity. For example such variants may include one or more amino acid deletions, substitutions, or additions (or combinations thereof), such as 1-50 of such changes (such as 1-40, 1-30, 1-20, or 1-10 of such changes). In certain examples, Mfn1 has at least 80%, at least 85%, at least 90%, at least 95%, at least 97%, at least 98% or at least 99% sequence identity to such sequences (such as SEQ ID NO: 100), and retains Mfn1 activity.

One of skill will realize that variants of ERRα, ERRγ, and/or Mfn1 proteins can be used, such as a variant containing conservative amino acid substitutions. Such conservative variants will retain critical amino acid residues necessary for ERRα, ERRγ, and/or Mfn1 activity, and will retain the charge characteristics of the residues in order to preserve the low pI and low toxicity of the molecules. Amino acid substitutions (such as at most one, at most two, at most three, at most four, at most five, or at most 10 amino acid substitutions, such as 1 to 10 or 1 to 5 conservative substitutions) can be made in an ERRα, ERRγ, and/or Mfn1 protein sequence to increase yield. Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

An ERRα, ERRγ, and/or Mfn1 protein can be derivatized or linked to another molecule (such as another peptide or protein). For example, the ERRα, ERRγ, and/or Mfn1 protein can be functionally linked (by chemical coupling, genetic fusion, noncovalent association or otherwise) to one or more other molecular entities, such as an antibody, a detection agent, or a pharmaceutical agent.

Methods of making proteins are routine in the art, for example by recombinant molecular biology methods or by chemical peptide synthesis. In one example, an ERRα, ERRγ, and/or Mfn1 protein is expressed in a cell from a vector encoding the protein. In some examples, the expression vector encoding ERRα, ERRγ, and/or Mfn1 also encodes a selectable marker. In some examples, the sequence encoding ERRα, ERRγ, and/or Mfn1 also encodes a purification tag sequence (such as a His-tag, β-globin-tag or glutathione S-transferase-(GST) tag) at the N- or C-terminus of ERRα, ERRγ, and/or Mfn1, to assist in purification of the protein.

For example, expression of nucleic acids encoding ERRα, ERRγ, and/or Mfn1 proteins can be achieved by operably linking the ERRα, ERRγ, and/or Mfn1 DNA or cDNA to a promoter (which is either constitutive or inducible), followed by incorporation into an expression cassette. The promoter can be any promoter, including a cytomegalovirus promoter and a human T cell lymphotrophic virus promoter (HTLV)-1. Optionally, an enhancer, such as a cytomegalovirus enhancer, is included in the construct. The cassettes can be suitable for replication and integration in either prokaryotes (such as E. coli) or eukaryotes (such as yeast or a mammalian cell). Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of ERRα, ERRγ, and/or Mfn1, expression cassettes can include a strong promoter to direct transcription, a ribosome binding site for translational initiation (internal ribosomal binding sequences), and a transcription/translation terminator. Exemplary control sequences include the T7, trp, lac, tac, trc, or lambda promoters, the control region of fd coat protein, a ribosome binding site, and can include a transcription termination signal. For eukaryotic cells, the control sequences can include a promoter and/or an enhancer derived from, for example, an immunoglobulin gene, HTLV, SV40, polyoma, adenovirus, retrovirus, baculovirus, simian virus, promoters derived from the promoter for 3-phosphoglycerate kinase, the promoters of yeast acid phosphatase, the promoter of the yeast alpha-mating factors or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding EERγ, and a second foreign DNA molecule encoding a selectable phenotype, such as the herpes simplex thymidine kinase gene. Another method is to use a eukaryotic viral vector, such as simian virus 40 (SV40), retrovirus, adenovirus, adeno-associated virus, Herpes virus, or bovine papilloma virus, to transiently infect or transform eukaryotic cells and express the protein (see for example, Eukaryotic Viral Vectors, Cold Spring Harbor Laboratory, Gluzman ed., 1982). One can readily use an expression system, such as plasmids and vectors, to produce proteins in cells including higher eukaryotic cells such as the COS, CHO, HeLa, fibroblast cell lines, lymphoblast cell lines, and myeloma cell lines.

Once expressed, the recombinant ERRα, ERRγ, and/or Mfn1 protein can be purified according to standard procedures of the art, including ammonium sulfate precipitation, affinity columns, column chromatography, and the like (see, generally, R. Scopes, PROTEIN PURIFICATION, Springer-Verlag, N.Y., 1982). The recovered ERRα, ERRγ, and/or Mfn1 protein need not be 100% pure. Once purified, partially or to homogeneity as desired, the ERRα, ERRγ, and/or Mfn1protein can be used therapeutically.

Modifications can be made to a nucleic acid encoding ERRα, ERRγ, and/or Mfn1 without diminishing its biological activity. Some modifications can be made to facilitate the cloning, expression, or incorporation of ERRα, ERRγ, and/or Mfn1 into a fusion protein. Such modifications are well known and include, for example, termination codons, a methionine added at the amino terminus to provide an initiation, site, additional amino acids placed on either terminus to create conveniently located restriction sites, or additional amino acids (such as poly His) to aid in purification steps.

In one example, ERRα, ERRγ, and/or Mfn1 protein is synthesized by condensation of the amino and carboxyl termini of shorter fragments. Methods of forming peptide bonds by activation of a carboxyl terminal end (such as by the use of the coupling reagent N, N′-dicylohexylcarbodimide) are well known.

Administration and Expression of Nucleic Acid Molecules in a Subject

In one example, ERRα, ERRγ, and/or Mfn1 activity is increased by administering to the subject a nucleic acid molecule encoding an ERRα, ERRγ, and/or Mfn1 protein, respectively. ERRα, ERRγ, and Mfn1 coding sequences are known. For example, GenBank® Accession Nos. NM_001134285.1, AY388461, AF058291.1 and NM_011935.2 disclose exemplary ERRγ nucleic acid sequences. In addition, GenBank® Accession Nos. NM_001282450 and NM_007953 disclose exemplary ERRα nucleic acid sequences. In addition, GenBank® Accession Nos. NM_033540.2, NM_024200.4, NM_001206508.1 and NM_138976.1 disclose exemplary Mfn1 nucleic acid sequences. However, one skilled in the art will appreciate that variations of such sequences can also encode a protein with ERRα, ERRγ, and/or Mfn1 activity. For example such variants may include encode a protein with one or more amino acid deletions, substitutions, or additions (or combinations thereof), such as 1-50 of such changes (such as 1-40, 1-30, 1-20, or 1-10 of such changes). In certain examples, an ERRγ coding sequence has at least 80%, at least 85%, at least 90%, at least 95%, at least 97%, at least 98% or at least 99% sequence identity to such sequences (such as SEQ ID NO: 97), and encodes a protein having ERRγ activity. In certain examples, an ERRα coding sequence has at least 80%, at least 85%, at least 90%, at least 95%, at least 97%, at least 98% or at least 99% sequence identity to such sequences (such as SEQ ID NO: 95), and encodes a protein having ERRα activity. In certain examples, a Mfn1 coding sequence has at least 80%, at least 85%, at least 90%, at least 95%, at least 97%, at least 98% or at least 99% sequence identity to such sequences (such as SEQ ID NO: 99), and encodes a protein having Mfn1 activity. One of skill in the art can readily use the genetic code to construct a variety of functionally equivalent nucleic acids, such as nucleic acids which differ in sequence but which encode the same ERRα, ERRγ, or Mfn1 protein sequence.

Nucleic acid sequences encoding an ERRα, ERRγ, or Mfn1 protein can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template. One of skill will recognize that longer sequences may be obtained by the ligation of shorter sequences.

Exemplary ERRα, ERRγ, and Mfn1 nucleic acids can be prepared by routine cloning techniques. Examples of appropriate cloning and sequencing techniques, and instructions sufficient to direct persons of skill through many cloning exercises are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources. Nucleic acids can also be prepared by amplification methods. A wide variety of cloning methods, host cells, and in vitro amplification methodologies are well known.

In some examples, it may only be necessary to introduce the ERRα, ERRγ, or Mfn1 genetic or protein elements into certain cells or tissues. For example, introducing ERRα, ERRγ, and/or Mfn1 into only the muscle, such as skeletal or cardiac muscle (or even a particular muscle), may be sufficient. However, in some instances, it may be more therapeutically effective and simple to treat all of the patient's cells, or more broadly disseminate the ERRα, ERRγ, and/or Mfn1 nucleic acid or protein, for example by intravascular administration.

Nucleic acids encoding ERRα, ERRγ, and/or Mfn1 can be introduced into the cells of a subject using routine methods, such as by using recombinant viruses (e.g., viral vectors) or by using naked DNA or DNA complexes (non-viral methods). Thus, in some embodiments, a method of increasing ERRα, ERRγ, and/or Mfn activity in persons suffering from, or at risk for, a mitochondrial disease, and/or cardiac disease, is achieved by introducing a nucleic acid molecule coding for ERRα, ERRγ, and/or Mfn1 into the subject. A general strategy for transferring genes into donor cells is disclosed in U.S. Pat. No. 5,529,774. The nucleic acid encoding ERRα, ERRγ, and/or Mfn1 can be administered to the subject by any method which allows the recombinant nucleic acid to reach the appropriate cells. Exemplary methods include injection, infusion, deposition, implantation, and topical administration. Injections can be intradermal, intramuscular, iv, or subcutaneous.

In one example, an ERRα, ERRγ, and/or Mfn1 coding sequence is introduced into a subject in a non-infectious form, such as naked DNA or liposome encapsulated DNA. Such molecules can be introduced by injection (such as intramuscular, iv, ip, pneumatic injection, or a gene gun), or other routine methods (such as oral or nasal). In one example, ERRα, ERRγ, and/or Mfn1γ coding sequence is part of a lipoplex, dendrimer, or inorganic nanoparticle to assist in its delivery.

In one example, viral vectors are used. Generally, such methods include cloning an ERRα, ERRγ, and/or Mfn1 coding sequence into a viral expression vector, and that vector is then introduced into the subject to be treated. The virus infects the cells, and produces the ERRα, ERRγ, and/or Mfn1 protein sequence in vivo, where it has its desired therapeutic effect. The nucleic acid sequence encoding ERRα, ERRγ, and/or Mfn1 can be placed under the control of a suitable promoter. Suitable promoters which may be employed include, but are not limited to, the gene's native promoter; retroviral LTR promoter; adenoviral promoters, such as the adenoviral major late promoter; the cytomegalovirus (CMV) promoter; the Rous Sarcoma Virus (RSV) promoter; inducible promoters, such as the MMTV promoter; the metallothionein promoter; heat shock promoters; the albumin promoter; the histone promoter; the β-actin promoter; TK promoters; B19 parvovirus promoters; and the ApoAI promoter.

Exemplary viral vectors include, but are not limited to: pox viruses, recombinant vacciniavirus, retroviruses (such as lentivirus), replication-deficient adenovirus strains, adeno-associated virus, herpes simplex virus, or poliovirus.

Adenoviral vectors may include essentially the complete adenoviral genome. Alternatively, the adenoviral vector may be a modified adenoviral vector in which at least a portion of the adenoviral genome has been deleted. In one embodiment, the vector includes an adenoviral 5′ ITR; an adenoviral 3′ ITR; an adenoviral encapsidation signal; a DNA sequence encoding a therapeutic agent such as EDA1-II, dl or DL; and a promoter for expressing the DNA sequence encoding a therapeutic agent. The vector is free of at least the majority of adenoviral E1 and E3 DNA sequences, but is not necessarily free of all of the E2 and E4 DNA sequences, and DNA sequences encoding adenoviral proteins transcribed by the adenoviral major late promoter. Such a vector may be constructed according to standard techniques, using a shuttle plasmid which contains, beginning at the 5′ end, an adenoviral 5′ ITR, an adenoviral encapsidation signal, and an E1a enhancer sequence; a promoter (which may be an adenoviral promoter or a foreign promoter); a tripartite leader sequence, a multiple cloning site (which may be as herein described); a poly A signal; and a DNA segment which corresponds to a segment of the adenoviral genome. The DNA segment serves as a substrate for homologous recombination with a modified or mutated adenovirus, and may encompass, for example, a segment of the adenovirus 5′ genome no longer than from base 3329 to base 6246. The plasmid may also include a selectable marker and an origin of replication. The origin of replication may be a bacterial origin of replication. A desired DNA sequence encoding a therapeutic agent may be inserted into the multiple cloning site of the plasmid. The plasmid may be used to produce an adenoviral vector by homologous recombination with a modified or mutated adenovirus in which at least the majority of the E1 and E3 adenoviral DNA sequences have been deleted. Homologous recombination may be effected through co-transfection of the plasmid vector and the modified adenovirus into a helper cell line, such as 293 cells, by CaPO₄ precipitation. The homologous recombination produces a recombinant adenoviral vector which includes DNA sequences derived from the shuttle plasmid between the Not I site and the homologous recombination fragment, and DNA derived from the E1 and E3 deleted adenovirus between the homologous recombination fragment and the 3′ ITR.

In one embodiment, the viral vector is a retroviral vector. Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, lentivirus, myeloproliferative sarcoma virus, and mammary tumor virus. The vector can be a replication defective retrovirus particle. Retroviral vectors are useful as agents to effect retroviral-mediated gene transfer into eukaryotic cells. Retroviral vectors are generally constructed such that the majority of sequences coding for the structural genes of the virus are deleted and replaced by the gene(s) of interest. Most often, the structural genes (e.g., gag, pol, and env), are removed from the retroviral backbone using genetic engineering techniques known in the art. An ERRα, ERRγ, and/or Mfn1 coding sequence can be incorporated into a proviral backbone using routine methods. In the most straightforward constructions, the structural genes of the retrovirus are replaced by a ERRα, ERRγ, and/or Mfn1 gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter. Alternatively, two genes may be expressed from a single promoter by the use of an Internal Ribosome Entry Site.

In one example, the viral vector is an adeno-associated virus (AAV). Gene therapy vectors using AAV can infect both dividing and non-dividing cells and persist in an extrachromosomal state without integrating into the genome of the host cell. In some examples, the rep and cap are removed from the DNA of the AAV. The ERRα, ERRγ, and/or Mfn1 coding sequence together with a promoter to drive transcription is inserted between the inverted terminal repeats (ITR) that aid in concatamer formation in the nucleus after the single-stranded vector DNA is converted by host cell DNA polymerase complexes into double-stranded DNA.

The viral particles are administered in an amount effective to produce a therapeutic effect in a host. The exact dosage of viral particles to be administered is dependent upon a variety of factors, including the age, weight, and sex of the patient to be treated, and the nature and extent of the disease or disorder to be treated. The viral particles may be administered as part of a preparation having a titer of viral particles of at least 1×10⁵ pfu/ml, at least 1×10⁶ pfu/ml, at least 1×10⁷ pfu/ml, at least 1×10⁸ pfu/ml, at least 1×10⁹ pfu/ml, or at least 1×10¹⁰ pfu/ml, and in some examples not exceeding 2×10¹¹ pfu/ml. The viral particles can be administered in combination with a pharmaceutically acceptable carrier, for example in a volume up to 10 ml. The pharmaceutically acceptable carrier may be, for example, a liquid carrier such as a saline solution, protamine sulfate or Polybrene.

Agonists

An ERRγ agonist is an agent that induces or increases ERRγ activity or expression. Agonists of ERRγ are commercially available, and can be generated using routine methods. In some examples, the agonist is an agonist of ERRγ, but not ERRα or ERRβ. In some examples, the agonist is an agonist of ERRγ, as well as of ERRα and/or ERRβ3.

ERRγ agonists are known in the art, and additional ERRγ agonists can be identified using known methods (e.g., see Zuercher et al., 2005, J. Med. Chem. 48(9):3107-9; Coward et al. 2001, Proc Natl Acad Sci US A. 8(15):8880-4; and Zhou et al., 1998, Mol. Endocrin. 12:1594-1604).

For example, phenolic acyl hydrazones GSK4716 (e.g., Santa Cruz Catalog #sc-203986) and GSK9089 (also known as DY131, see for example, U.S. Pat. No. 7,544,838) (N-[(E)-[4-(diethylamino)phenyl]methylideneamino]-4-hydroxybenzamide; e.g., Tocris Bioscience Catalog #2266 or Santa Cruz Catalog # sc203571) are agonists of ERRβ and ERRγ.

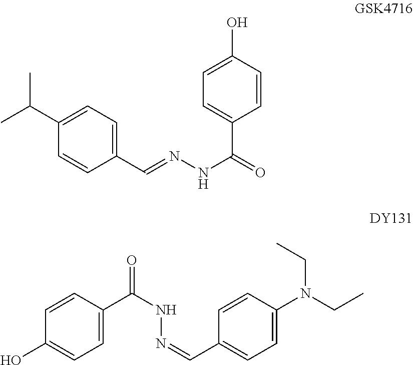 [SMILES: CC(C)c1ccc(/C=N/NC(=O)c2ccc(O)cc2)cc1 ; CCN(CC)c1ccc(/C=N\NC(=O)c2ccc(O)cc2)cc1]

Kim et al. (J. Comb. Chem. 11:928-37, 2009) disclose a screening assay for agonists of ERRγ derived from GSK4716. Such a screening method can also be used to identify other agonists of ERRγ. E6 was discovered as being selective for ERRγ but not ERRα and β.

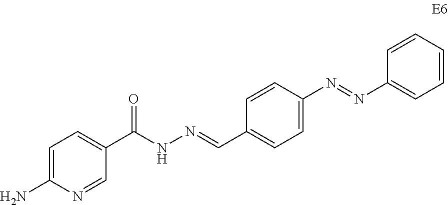 [SMILES: Nc1ccc(C(=O)N/N=C/c2ccc(/N=N/c3ccccc3)cc2)cn1]

U.S. Pat. Nos. 7,544,838 and 8,044,241 also provide ERRγ agonists that can be used with the disclosed methods, such as DY131. In addition, DY159, DY162, DY163 and DY164 were also observed to activate ERRγ (and ERRα and β), for example in the presence of PGC-1a.

US Patent Application Publication Nos. 2011/0218196 and 2009/0281191 also provide ERRγ agonists that can be used with the disclosed methods.

In one example, the ERRγ agonist is GSK5182 An ERRα agonist is an agent that induces or increases ERRα activity or expression. Agonists of ERRαγ are commercially available, and can be generated using routine methods. Exemplary ERRα agonists are provided in US Patent Publication No. 20150011544, herein incorporated by reference. In one example, the ERRα agonist is

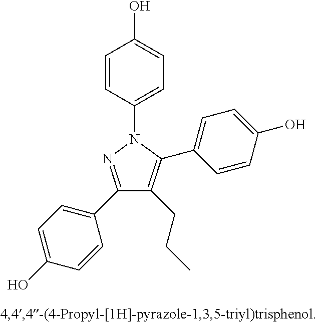 [SMILES: CCCc1c(-c2ccc(O)cc2)nn(-c2ccc(O)cc2)c1-c1ccc(O)cc1]

Patch et al. (2011. J Med Chem 54:788-808) also provide ERRα agonists that can be used with the disclosed methods.

Pharmaceutical Compositions

Pharmaceutical compositions that include an ERRα, ERRγ, and/or Mfn1 protein (such as a protein sequence in SEQ ID NO: 96, 98 or 100, or a nucleic acid encoding such), or an ERRα, ERRγ, and/or Mfn1 agonist, can be formulated with an appropriate pharmaceutically acceptable carrier, depending upon the particular mode of administration chosen. Such compositions can be used in the methods or be part of the kits provided herein.

In some embodiments, the pharmaceutical composition consists essentially of a ERRα, ERRγ, and/or Mfn1 protein (such as a protein having at least 90%, at least 92%, at least 95%, at least 96%, at least 97%, at least 98%, at least 99% or 100% sequence identity to SEQ ID NO: 96, 98 or 100, or a nucleic acid encoding such a protein) and a pharmaceutically acceptable carrier. In some embodiments, the pharmaceutical composition consists essentially of a ERRα, ERRγ, and/or Mfn1 agonist and a pharmaceutically acceptable carrier. In these embodiments, additional therapeutically effective agents are not included in the compositions.

In other embodiments, the pharmaceutical composition includes an ERRα, ERRγ, and/or Mfn1 protein (such as a protein having at least 90%, at least 92%, at least 95%, at least 96%, at least 97%, at least 98%, at least 99% or 100% sequence identity to SEQ ID NO: 96, 98 or 100, or a nucleic acid encoding such a protein), or an ERRα, ERRγ, and/or Mfn1 agonist, and a pharmaceutically acceptable carrier. Additional therapeutic agents, such as agents for the treatment of heart failure, bradycardia and/or cardiomyopathy, can be included. Thus, the pharmaceutical compositions used in the disclosed methods (or part of the disclosed kits) can include a therapeutically effective amount of another agent. Examples of such agents include, without limitation, an agent that balances electrolytes (e.g., aldosterone antagonists such as spironolactone or eplerenone), an agent that prevents arrhythmias (antiarrhythmics such as amiodarone, bepridil hydrochloride, disopyramide, dofetilide, dronedarone, flecaninide, ibutilide, lidocaine, procainamide, propafenone, propranolol, quinidine, sotalol, and tocanide); an agent that lowers blood pressure (e.g., ACE inhibitors [such as benazepril, captopril, enalapril, fosinopril, lisinopril, moexipril, perindopril, quinapril, ramipril, and trandolapril]; angiotensin II receptor blockers [such as losartan, candesartan, valsartan, irbesartan, telmisartan, eprosatan, olmesartan, azilsartan, and fimasartan), beta blockers [such as acebutolol, atenolol, betaxolol, bisoprolol, celiprolol, esmolol, metoprolol, landiolol, labetalol, carvedilol, and nebivolol], and calcium channel blockers [such as dihydropyridines (e.g., amlodipine, barnidipine, efonidipine) and non-dihydropyridines (e.g., phenylalkylamines such as verapamil, gallopamil, and fendiline; and benzothiazepines such as ditiazem)], an agent that prevents blood clots from forming (e.g., anticoagulants such as dabigatran, rivaroxaban, apixaban, edoxban, coumarin, acenocoumarol, phenprocoumon, atromentin, phenindione, heparin, rivaroxaban, apixaban edoxaban, acenocoumarol, phenprocoumon, atromentin, and phenindione), an agent that reduces inflammation (e.g., corticosteroids such as corticosterone, cortisone, aldosterone), an agent that removes excess sodium (e.g., diuretics such as thiazides (e.g., hydrochlorothiazide), acetazolamide, methazolamide, lasix, bumex, aldactone, dyrenium, and mannitol), an agent that decreases heart rate (e.g., beta blockers [such as acebutolol, atenolol, betaxolol, bisoprolol, celiprolol, esmolol, metoprolol, landiolol, labetalol, carvedilol, and nebivolol], calcium channel blockers [such as dihydropyridines (e.g., amlodipine, barnidipine, efonidipine) and non-dihydropyridines (e.g., phenylalkylamines such as verapamil, gallopamil, and fendiline; and benzothiazepines such as ditiazem)], and digoxin), isosorbide dinitrate/hydralazine hydrochloride, or combinations thereof (such as 1, 2, 3, 4 or 5 of such agents).

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, an ERRα, ERRγ, and/or Mfn1 protein (such as a protein sequence in SEQ ID NO: 96, 98 or 100, or a nucleic acid encoding such), or an ERRα, ERRγ, and/or Mfn1 agonist, is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other embodiments, an ERRα, ERRγ, and/or Mfn1 protein (such as a protein having at least 90%, at least 92%, at least 95%, at least 96%, at least 97%, at least 98%, at least 99% or 100% sequence identity to SEQ ID NO: 96, 98 or 100, or a nucleic acid encoding such a protein), or an ERRα, ERRγ, and/or Mfn1 agonist, is included in a nanodispersion system. Nanodispersion systems and methods for producing such nanodispersions are well known to one of skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006. With regard to the administration of nucleic acids, one approach to administration of nucleic acids is direct treatment with plasmid DNA, such as with a mammalian expression plasmid. As described above, a nucleotide sequence encoding an ERRα, ERRγ, and/or Mfn1 protein (such as a protein having at least 90%, at least 92%, at least 95%, at least 96%, at least 97%, at least 98%, at least 99% or 100% sequence identity to SEQ ID NO: 96, 98 or 100) can be placed under the control of a promoter to increase expression of the protein.

Many types of release delivery systems are available and known. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an ERRα, ERRγ, and/or Mfn1 protein (such as a protein having at least 90%, at least 92%, at least 95%, at least 96%, at least 97%, at least 98%, at least 99% or 100% sequence identity to SEQ ID NO: 96, 98 or 100, or a nucleic acid encoding such a protein), is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant may be particularly suitable for treatment of chronic conditions, such as heart failure, bradycardia and/or cardiomyopathy. Long-term release, as used herein, means that the implant is constructed and arranged to deliver therapeutic levels of the active ingredient for at least 30 days, and such as at least 60 days. Long-term sustained release implants are well known to those of ordinary skill in the art and include some of the release systems described above. These systems have been described for use with nucleic acids (see U.S. Pat. No. 6,218,371). For use in vivo, nucleic acids and peptides are preferably relatively resistant to degradation (such as via endo- and exo-nucleases). Thus, modifications of an ERRα, ERRγ, and/or Mfn1 protein, such as the inclusion of a C-terminal amide, can be made.

The dosage form of the pharmaceutical composition can be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

### Example 1

### Materials and Methods

Animal Studies.

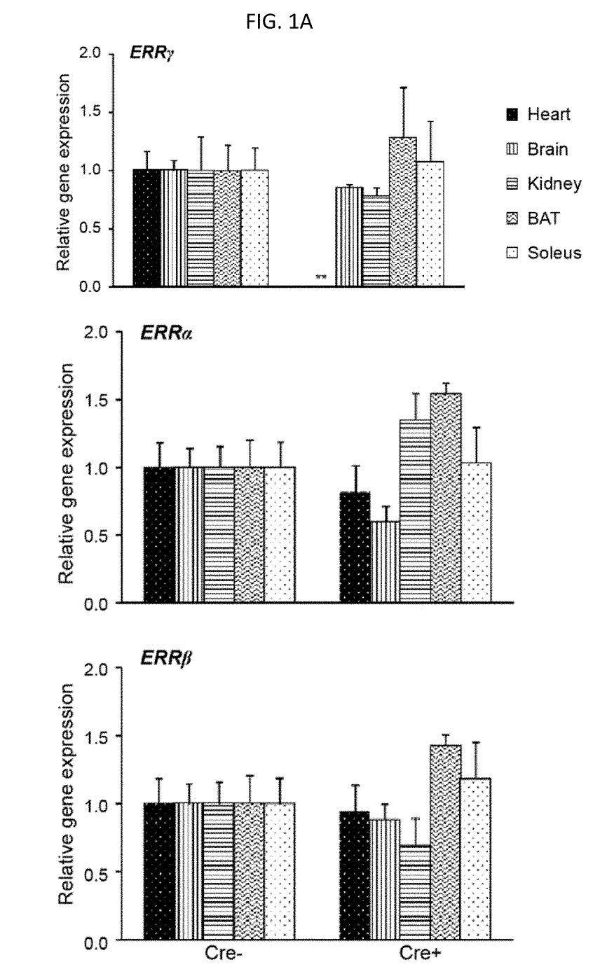

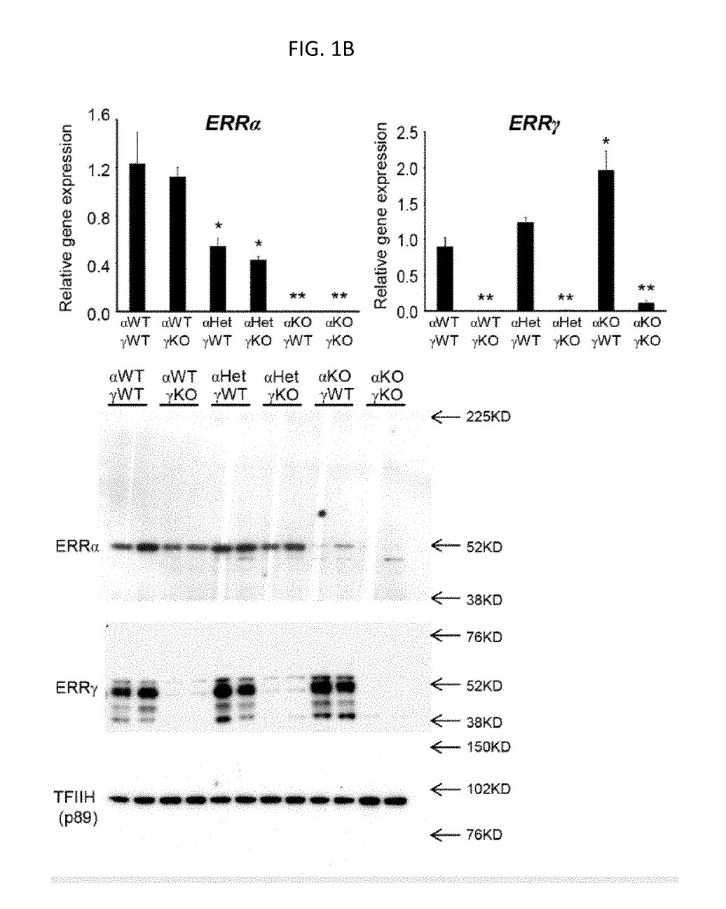

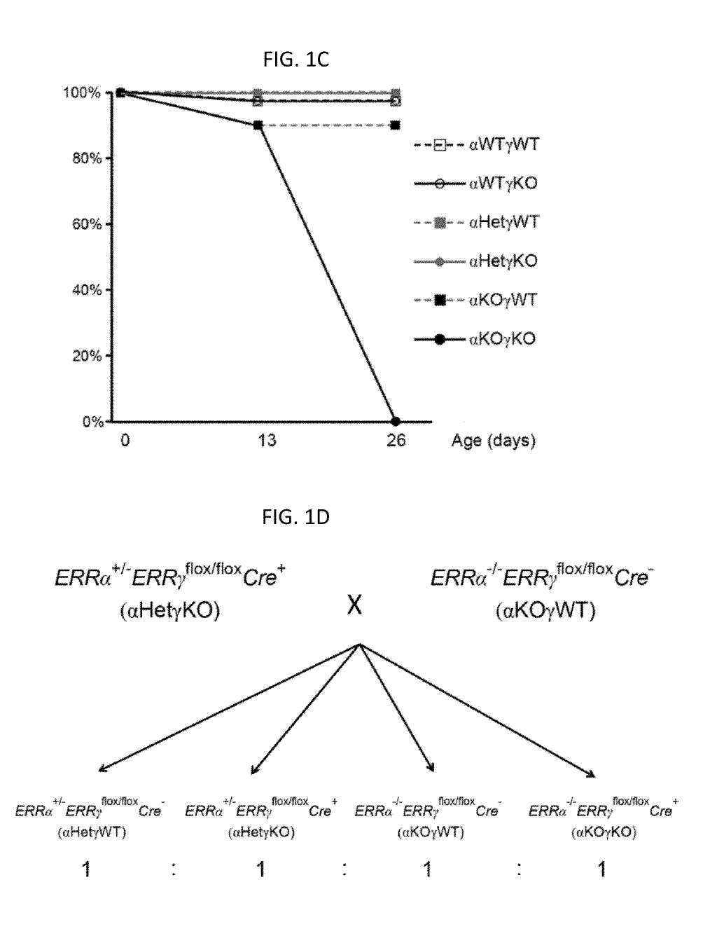

Mice were maintained in a temperature- and light-controlled environment with ad libitum access to water. Mice in holding cages (after weaning) received a standard chow diet (Lab Diet 5L0D, 58% calories from carbohydrate, 13.5% calories from fat and 28.5% calories from proteins), and breeder mice and their pups before weaning received a breeder diet (Lab Diet 5058, 55% calories from carbohydrate, 22% calories from fat and 23% calories from proteins). ERRα KO and ERRγ^{flox/flox} (exon 2 is floxed) mice were previously described (Luo et al., 2003. Mol Cell Biol 23:7947-7956; Gan et al., 2013. J Clin Invest 123:2564-2575). All mice were backcrossed at least six generations to and maintained in the C57BL6/J background (JAX). For survival rate analysis, the breeding pairs were monitored daily for birth of pups. The first day new pups born was observed was deemed as PO and the pups were toe-clipped for identification and genotyping. Since toe-clipping could disturb the mother resulting in inadequate nurturing, all pups dying before P4 (all genotypes were represented in these pups) were excluded in the survival analysis (FIG. 1C). Both male and female pups were included in the study. All tissues were harvested between 2 and 5 pm of the day to avoid the impact of circadian rhythm.

Gene Expression Analysis.

Total RNA was isolated from mouse tissues or cells using RNAzol reagent (Molecular Research Center) according to the manufacturer's instructions. cDNA was synthesized from 1 μg total RNA using iScript cDNA synthesis kit (Bio-Rad) and mRNA levels quantified by real-time qRT-PCR using SYBR Green (Bio-Rad) (Wang et al., 2010. PLoS One 5; Pei et al., 2011. Nat Med 17:1466-1472). Relative mRNA levels were calculated using a standard curve and normalized to 36b4 mRNA levels in the same samples. The qPCR primer sequences are listed in Table 1.

TABLE 1
Sequences of mouse qPCR Primers used in ChIP,
mtDNA and gene expression analysis

| ChIP primers | Sequence (SEQ ID NO:) |
|---|---|
| Mfn1 For | TGCATGTTTCACCACAGTTTC (1) |
| Mfn1 Rev | GTAGCTCACAACCACCTGTAA (2) |
| Mfn2 For | TCCAATGCAGTATCCCAGTTC (3) |
| Mfn2 Rev | CCAGGACATTCAGGACATGATTA (4) |
| Kcnq1 For | CCCGCAGCTAATTGCTTTAGA (5) |
| Kcnql Rev | CATAAACAGACCTCTGGACAACC (6) |
| Kcnh2 For | CTGCCAGATGACCTTGAGTG (7) |
| Kcnh2 Rev | GCCCTGTAGTTTATCACCTTGT (8) |
| Tnnt2 For | CAAAGGGAATTATGTTCTGGGAAA (9) |
| Tnnt2 Rev | GGAAAGAGTAAGGTCTCGGTATG (10) |
| Tnnc1 For | CCCACACACCTGTAACCC (11) |
| Tnnc1 Rev | TGCTGAAAGCTGAGACCATAC (12) |

mtDNA/nDNA analysis primers

| | |
|---|---|
| Cytb For | CATTTATTATCGCGGCCCTA (13) |
| Cytb Rev | TGTTGGGTTGTTTGATCCTG (14) |
| Cox1 For | TGCTAGCCGCAGGCATTACT (15) |
| Cox1 Rev | CGGGATCAAAGAAAGTTGTGT (16) |
| Glucagon For | CAGGGCCATCTCAGAACC (17) |
| Glucagon Rev | GCTATTGGAAAGCCTCTTGC (18) |
| Globin For | GAAGCGATTCTAGGGAGCAG (19) |
| Globin Rev | GGAGCAGCGATTCTGAGTAGA (20) |

qRT-PCR primers

| | |
|---|---|
| ERRα For | CTCAGCTCTCTACCCAAACGC (21) |
| ERRα Rev | CCGCTTGGTGATCTCACACTC (22) |
| ERRβ For | CAGATCGGGAGCTTGTGTTC (23) |
| ERRβ Rev | TGGTCCCCAAGTGTCAGACT (24) |
| ERRγ For | GAATCTTTTTCCCTGCACTACGA (25) |
| ERRγ Rev | GCTGGAATCAATGTGTCGATCTT (26) |
| ANP For | GCTTCCAGGCCATATTGGAG (27) |
| ANP Rev | GGGGGCATGACCTCATCTT (28) |
| BNP For | GAGGTCACTCCTATCCTCTGG (29) |
| BNP Rev | GCCATTTCCTCCGACTTTTCTC (30) |
| CS For | GGACAATTTTCCAACCAATCTGC (31) |
| CS Rev | TCGGTTCATTCCCTCTGCATA (32) |
| Ndufa4 For | TCCCAGCTTGATTCCTCTCTT (33) |
| Ndufa4 Rev | GGGTTGTTCTTTCTGTCCCAG (34) |
| Sdhb For | CTGAATAAGTGCGGACCTATGG (35) |
| Sdhb Rev | AGTATTGCCTCCGTTGATGTTC (36) |
| Cox5a For | GCCGCTGTCTGTTCCATTC (37) |
| Cox5a Rev | GCATCAATGTCTGGCTTGTTGAA (38) |
| Atp5b For | ACGTCCAGTTCGATGAGGGAT (39) |
| Atp5b Rev | TTTCTGGCCTCTAACCAAGCC (40) |
| Cpt1b For | GCACACCAGGCAGTAGCTTT (41) |
| Cpt1b Rev | CAGGAGTTGATTCCAGACAGGTA (42) |
| Cpt2 For | CAGCACAGCATCGTACCCA (43) |
| Cpt2 Rev | TCCCAATGCCGTTCTCAAAAT (44) |
| Slc25a20 For | GACGAGCCGAAACCCATCAG (45) |
| Slc25a20 Rev | AGTCGGACCTTGACCGTGT (46) |
| Acadm For | AGGGTTTAGTTTTGAGTTGACGG (47) |
| Acadm Rev | CCCCGCTTTTGTCATATTCCG (48) |
| Echs1 For | AGCCTGTAGCTCACTGTTGTC (49) |
| Echs1 Rev | ATGTACTGAAAGTTAGCACCCG (50) |
| Hadha For | TGCATTTGCCGCAGCTTTAC (51) |
| Hadha Rev | GTTGGCCCAGATTTCGTTCA (52) |
| Gabpa For | CCAAGCACATTACGACCATTTC (53) |
| Gabpa Rev | CCGTGGACCAGCGTATAGGA (54) |
| Tfam For | CCACAGAACAGCTACCCAAATTT (55) |
| Tfam Rev | TCCACAGGGCTGCAATTTTC (56) |
| Mfn1 For | TGCAATCTTCGGCCAGTTACT (57) |
| Mfn1 Rev | CTCGGATGCTATTCGATCAAGTT (58) |
| Mfn2 For | AGAACTGGACCCGGTTACCA (59) |
| Mfn2 Rev | CACTTCGCTGATACCCCTGA (60) |
| Opa1 For | TGGAAAATGGTTCGAGAGTCAG (61) |
| Opa1 Rev | CATTCCGTCTCTAGGTTAAAGCG (62) |
| Drp1 For | CAGGAATTGTTACGGTTCCCTAA (63) |
| Drp1 Rev | CCTGAATTAACTTGTCCCGTGA (64) |
| Myh6 For | GCCCAGTACCTCCGAAAGTC (65) |
| Myh6 Rev | GCCTTAACATACTCCTCCTTGTC (66) |
| Actc1 For | CTGGATTCTGGCGATGGTGTA (67) |
| Actc1 Rev | CGGACAATTTCACGTTCAGCA (68) |
| Tnni3 For | TCTGCCAACTACCGAGCCTAT (69) |
| Tnni3 Rev | CTCTTCTGCCTCTCGTTCCAT (70) |
| Tnnt2 For | CAGAGGAGGCCAACGTAGAAG (71) |
| Tnnt2 Rev | CTCCATCGGGGATCTTGGGT (72) |
| Tnnc1 For | GCGGTAGAACAGTTGACAGAG (73) |
| Tnnc1 Rev | CCAGCTCCTTGGTGCTGAT (74) |
| Atp2a2 For | GAGAACGCTCACACAAAGACC (75) |
| Atp2a2 Rev | CAATTCGTTGGAGCCCCAT (76) |
| Pin For | AAAGTGCAATACCTCACTCGC (77) |
| Pin Rev | GGCATTTCAATAGTGGAGGCTC (78) |
| Ckmt2 For | ACACCCAGTGGCTATACCCTG (79) |
| Ckmt2 Rev | CCGTAGGATGCTTCATCACCC (80) |
| Mb For | CTGTTTAAGACTCACCCTGAGAC (81) |
| Mb Rev | GGTGCAACCATGCTTCTTCA (82) |
| Kcnq1 For | ACCTCATCGTGGTTGTAGCCT (83) |
| Kcnq1 Rev | GGATACCCCTGATAGCTGATGT (84) |
| Kcnh2 For | GTGCTGCCTGAGTATAAGCTG (85) |
| Kcnh2 Rev | CCGAGTACGGTGTGAAGACT (86) |
| Kcnj2 For | ATGGGCAGTGTGAGAACCAAC (87) |
| Kcnj2 Rev | TGGACTTTACTCTTGCCATTCC (88) |
| Scn5a For | ATGGCAAACTTCCTGTTACCTC (89) |
| Scn5a Rev | CCACGGGCTTGTTTTTCAGC (90) |
| Scn4b For | GGAACCGAGGCAATACTCAGG (91) |
| Scn4b Rev | CCGTTAATAGCGTAGATGGTGGT (92) |
| Cacna1c For | CCTGCTGGTGGTTAGCGTG (93) |
| Cacna1c Rev | TCTGCCTCCGTCTGTTTAGAA (94) |

Protein Analysis.

Nuclear extracts from mouse hearts were isolated and Western blot was performed as previously described (Pei et al., 2006. Nat Med 12:1048-1055). The primary antibodies used were: ERRα (Santa Cruz sc-32971), ERRγ (20) and TFIIH p89 (Santa Cruz sc-293).

Histology.

16 day old mice were euthanized and perfused with PBS and then 4% paraformaldehyde (1 ml/min for 5 min). The tissues were then dissected and fixed in 4% paraformaldehyde overnight. Tissues were embedded in paraffin and 5 am sections were used for H&E staining following standard procedures.

Electron Microscopy (EM) and Mitochondrial Size Analysis.

Electron microscopy was performed as previously described with minor modifications (Pei et al., 2011. Nat Med 17:1466-1472). Sectioned samples were imaged using a Jeol-1010 transmission electron microscope. For mitochondrial 2-dimensional size and perimeter analysis, 5 imaging fields (magnification of 10,000×) of longitudinal sections per genotype were used and each field contained at least 100 mitochondria. Image J freehand line tool was used to draw the outline of each mitochondria and added them as a “region of interest (ROI)” with the “ROI Manager” function in Image J. The size and perimeter were then calculated with the “measure” function.

mtDNA/nDNA Analysis.

To quantify the relative mtDNA/nDNA ratio, total DNA was isolated from cells or hearts using a DNA isolation kit (Qiagen) and used qPCR to quantify two mitochondria encoded genes (Cytb and Cox1) and two nucleus encoded genes (Glucagon and βGlobin). The relative quantities between Cytb and Cox1, and between Glucagon and βGlobin were highly comparable. The mtDNA/nDNA was calculated as the ratio between the average of Cytb/Cox1 and the average of Glucagon/βGlobin. The primer sequences were listed in Table 1.

Mitochondrial Enzyme Activity.

Hearts from 16 day old pups were collected, weighed and grinded in 20 volumes (v/w) of homogenization buffer (1 mM EDTA and 50 mM Triethanolamine in water) on ice. Citrate synthase (CS) enzymatic activity was determined by the change in absorbance of DTNB (Ellman's reagent) measured at 412 nm. Complex I (NADH dehydrogenase) activity was determined by the change in absorbance of NADH measured at 340 nm. Complex II (succinate dehydrogenase) activity was determined by the change in absorbance of DCIP measured at 600 nm. Complex IV (cytochrome c oxidase) enzymatic activity was determined by the change in absorbance of cytochrome c measured at 550 nm. All assays were performed in 96 well plates with the “Kinetic” function of a SpectraMax Paradigm Multi-Mode Microplate Detection Platform (Molecular Devices). The linear slopes (ΔOD/min) were calculated. The molar extinction coefficients used to calculate enzyme activity were 13.6 OD/mmol/cm (DTNB for CS), 6.22 OD/mmol/cm (NADH for complex I), 16.3 OD/mmol/cm (DCIP for complex II) and 29.5 OD/mmol/cm (cytochrome c for complex IV).

ChIP.

We performed ChIP in HL-1 cells as previously described (Pei et al., 2011. Nat Med 17:1466-1472). The antibodies used were: IgG (Santa Cruz sc-2027), ERR (Abcam ab16363), ERRγ (Dufour et al., 2007. Cell Metab 5:345-356) and acetylated histone H3 (Millipore 06-599, positive control). The ChIP qPCR primer sequences were listed in Table 1.

Transfections.

The cross-species conserved mouse Mfn2 promoter region (−722 to −503 bp) was PCR amplified and cloned into the BglII-XhoI sites, and the conserved mouse Mfn1 (+1035 to +1280 bp), Kcnq1 (+1192 bp to +1438 bp) and Kcnh2 (+1290 to +2485 bp) intron regions into the BamHI-SalI sites of the pGL4.10 basic luciferase reporter vector (Promega). 293 cells were transfected using Fugene HD (Promega) in 48-well plates with 100 ng luciferase reporter, 150 ng ERR expression vector or pcDNA3.1, and 10 ng Renilla control. Two days later cells were lysed. The luciferase activity was measured and normalized to Renilla control.

Mouse Embryonic Fibroblasts (MEFs).

Primary MEFs were derived from embryos of ERRα^{+/-}ERRγ^{+/-} (non-floxed strain) mouse breeding as previously described (Pei et al., 2011. Nat Med 17:1466-1472). MEFs were used within 5 passages for all the experiments.

Retroviral Infection.

To produce retrovirus, control mtDsRed or Mfn1 retroviral expression vectors were transfected with the packaging vector pCLEco (all gifts from David Chan, Caltech) into 293 cells. Virus containing medium was harvested 48 hours to 96 hours after transfection and concentrated with a Amicon Ultra-15 Centrifugal Filter (Mllipore). The virus was diluted with fresh medium and used to infect MEF cells with 5 μg/ml polybrene. Mitochondrial morphology and mtDNA/nDNA analysis was performed 3 and 12 days after the infection.

Mitochondrial Morphology Analysis.

MEFs were fixed with methanol for 10 minutes at −20° C. After incubating with an ATP5b antibody (A21351, Life Technologies) and fluorescence labeled secondary antibody (Jackson ImmunoResearch), the mitochondrial morphology was imaged using a Zeiss LSM710 confocal microscope with a 63× oil immersion lens (Wang et al., 2014. J Cell Sci 127:2483-2492). For mitochondrial size and perimeter analysis, 20-25 images per group were used. The background was subtracted with the “threshold” function of Image J to delete pixel intensities less than 70 (background), and then changed all the pixel intensity to 255 with “binary” function. The size and perimeter were set up in the “set measurements”, and the individual mitochondrial morphological parameters calculated with the “analyze particles” function.

Echocardiography.

15-16 day old mice were anesthetized with isoflurane (3% induction for 3 minutes, then maintained under 2% isoflurane). After their body weight was measured, the anesthetized mice were subjected to echocardiography using the Vevo2100 Imaging system (VisualSonics). The ambient temperature was maintained with a heating pad and lamp to avoid decrease in body temperature and bradycardia. The hearts were detected through Parasternal Long axis and A2 short axis Mid Ventricle view. 2D Measurements were performed to measure the LVAepid (LV epicardial area at end of diastole), LVAendd (LV endocardial area at end of diastole), LVAends (LV endocardial area at end of systole), LVLd (LV length from plane of the mitral valve to the apical endocardial surface during diastole) and LVLs (LV length from plane of the mitral valve to the apical endocardial surface during systole). M-Mode measurements were performed to measure the IVSd (thickness of the interventricular septum in diastole), LVPWd (LV posterior wall thickness in diastole), LVIDd (LV internal dimension in diastole) and LVIDs (LV internal dimension in systole). The other parameters were calculated as:

EDV=⅚(LVAendd×LVLd)

ESV=⅚(LVAends×LVLs)

SV=EDV−ESV

CO=SV×HR

EF=SV/EDV×100%

LVMass=1.05[(⅚LVAepid×(LVLd+\sqrt{LVAepid/3.14}−\sqrt{LVAendd/3.14}))−(⅚LVAendd×LVLd)]

LV relative wall thickness=(IVSd+LVPWd)/(IVSd+LVPWd+LVIDd)×100%

Electrocardiography (ECG).

ECG in conscious, ambulatory mice was recorded using ECGenie (Mouse Specifics) following manufacturer's instructions. At least 10 seconds of stable ECG recordings (more than 50 heart beats) were collected and the software generated the ensemble averaged signal that depicted the ECG morphology, from which P, Q, R, S and T were clearly identifiable. The heart rate, PR interval, QRS complex and QT interval were then calculated.

Ventricular Cardiomyocyte Isolation and Potassium Current Recordings.

12-16 day old mice were heparinized (50 units i.p.), anesthetized with pentobarbital (50 mg/kg) and their hearts were excised through a sternotomy (n=5-7 per group). The hearts were then mounted on a Langendorff apparatus and perfused with Ca²⁺-free Tyrode's solution for 6 minutes at 3.0-3.5 ml/min and a temperature of 36-37° C., followed by 12-15 minutes of perfusion with Ca²⁺-free Tyrode's solution containing collagenase plus protease. The atria were dissected away and the ventricles were kept in Ca²⁺-free Tyrode's solution with 1 mg/ml bovine serum albumin. Sections of ventricular tissue were then triturated gently with a Pasteur pipette to dissociate individual myocytes. Whole-cell patch clamp recording were obtained from single ventricular myocytes at room temperature (22-24° C.) within 12 hours of being isolated, as previously described (Wang et al., 2009. Proc Natl Acad Sci USA 106:7548-7552). Experiments were performed using an Axopatch 200B amplifier interfaced to a PC-computer via a 12-bit A/D interface running the pClamp 9.2 software. Potassium currents were elicited in response to 5-sec voltage steps, from −60 to +70 mV in 10 mV increments, from a holding potential of −80 mV. Each trial was preceded by a 20-ms depolarization to −20 mV to help eliminate contamination from voltage-gated inward Na⁺ currents that were not completely inhibited by tetrodotoxin. The bath solution contained (in mM): 136 NaCl, 4 KCl, 2 MgCl₂, 1 CaCl₂, 10 glucose and 10 HEPES; pH=7.4 with NaOH; tetrodotoxin (20 μM) and CdCl₂ (200 μM) were also added to suppress voltage-dependent Na⁺ and Ca²⁺ currents, respectively. The pipette solution contained (in mM): 135 KCl, 4 NaCl, 10 EGTA, 10 HEPES, 5 glucose, 3 MgATP and 0.5 Na₃GTP; pH=7.2 with KOH. Traces were digitized at 20 kHz and filtered at 5 kHz prior to storage for off-line analysis. Series resistances was compensated electronically (75-90%) resulting in uncompensated voltage errors less than 5 mV. Patch pipettes were fashioned from borosilicate glass and fire-polished to a final resistance of 2.0-2.5 MΩ.

Statistical Analysis:

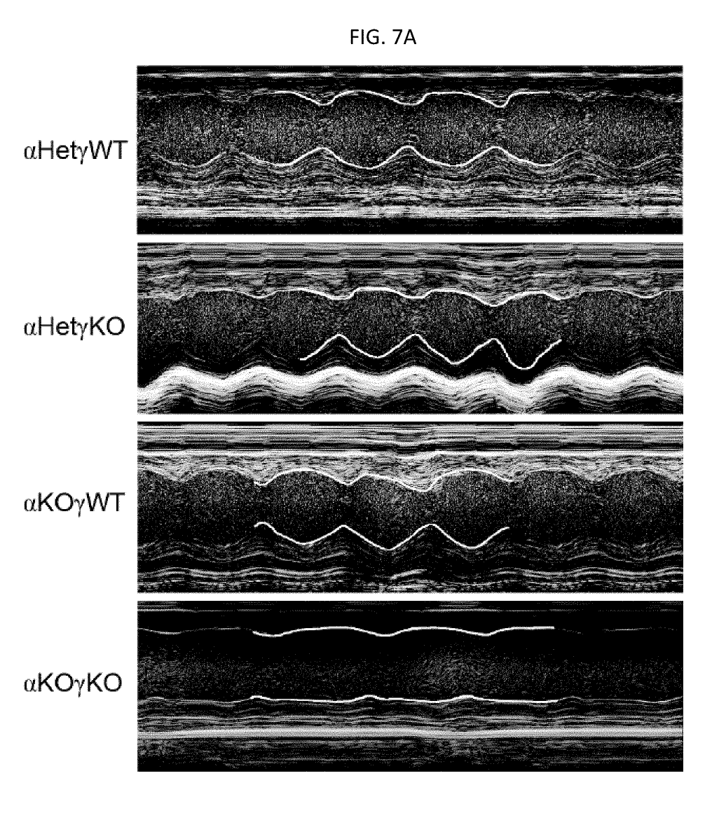

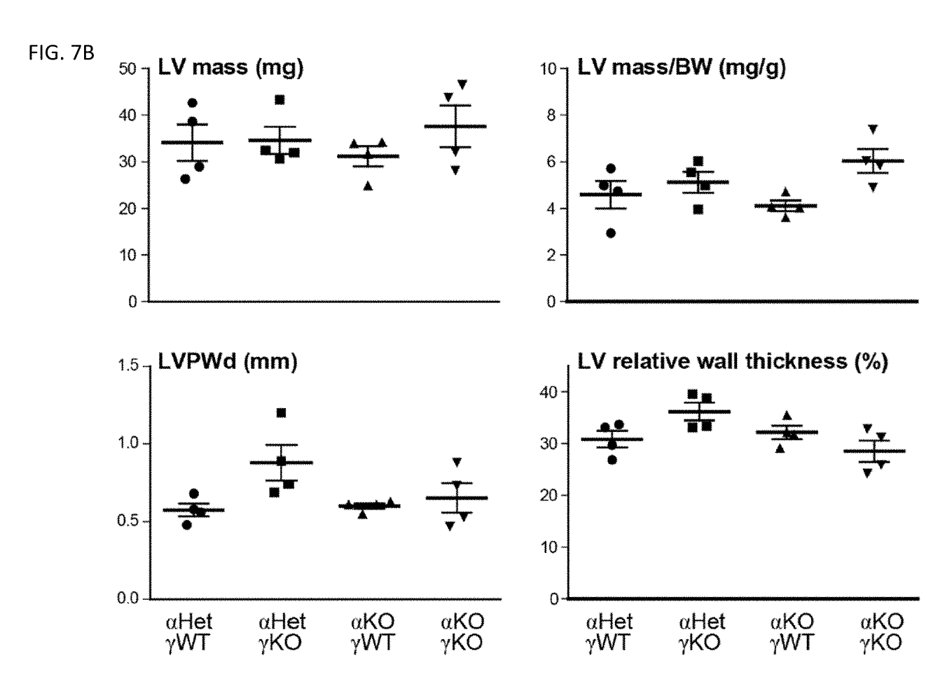

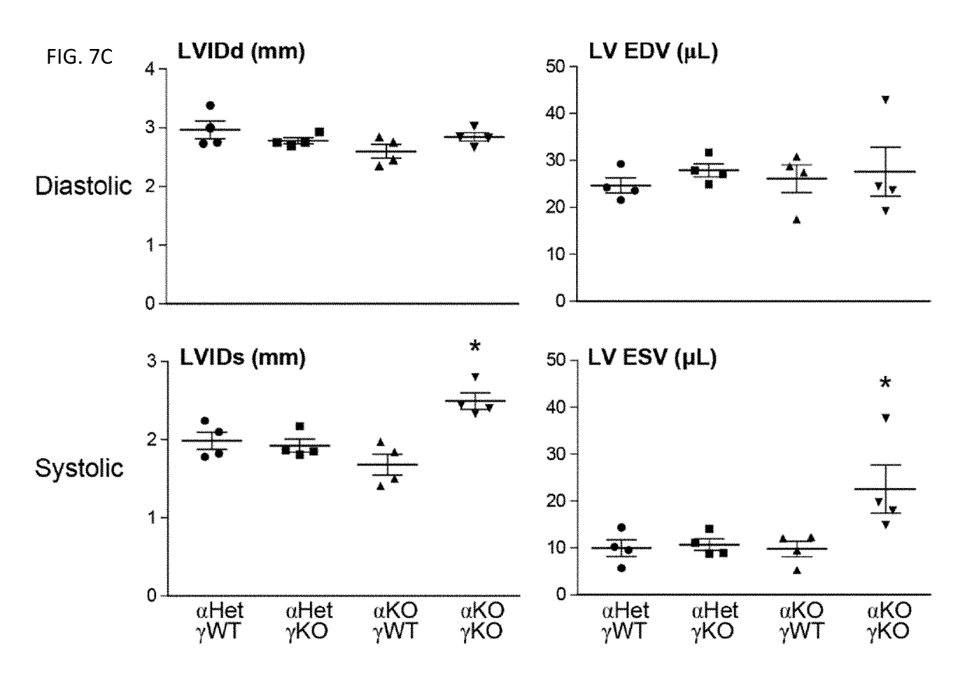

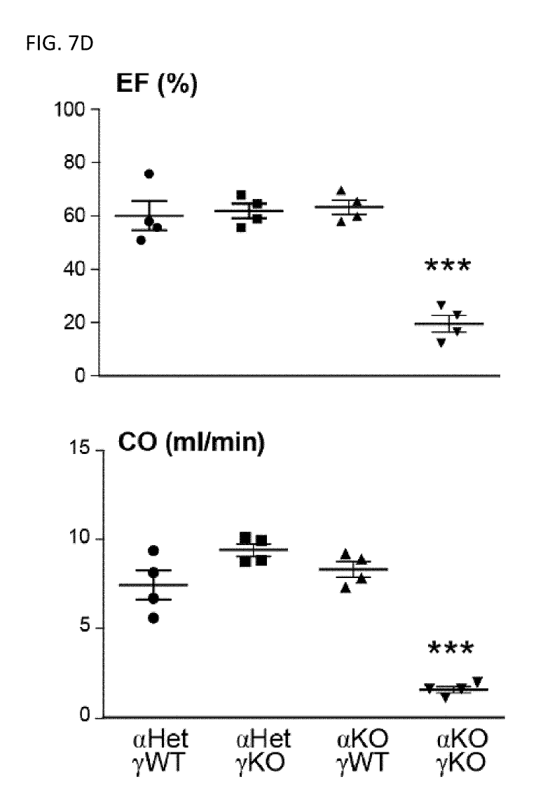

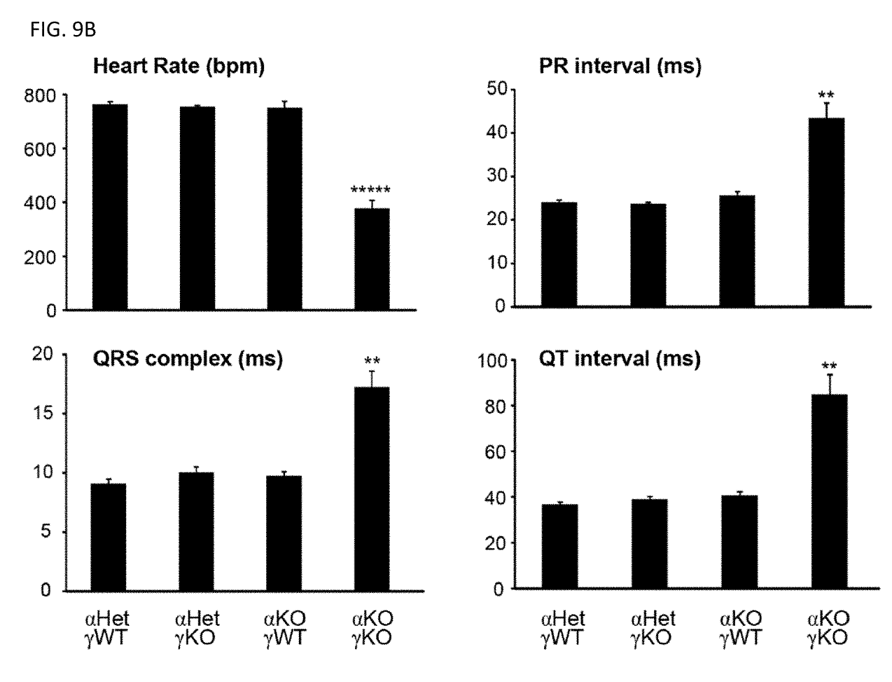

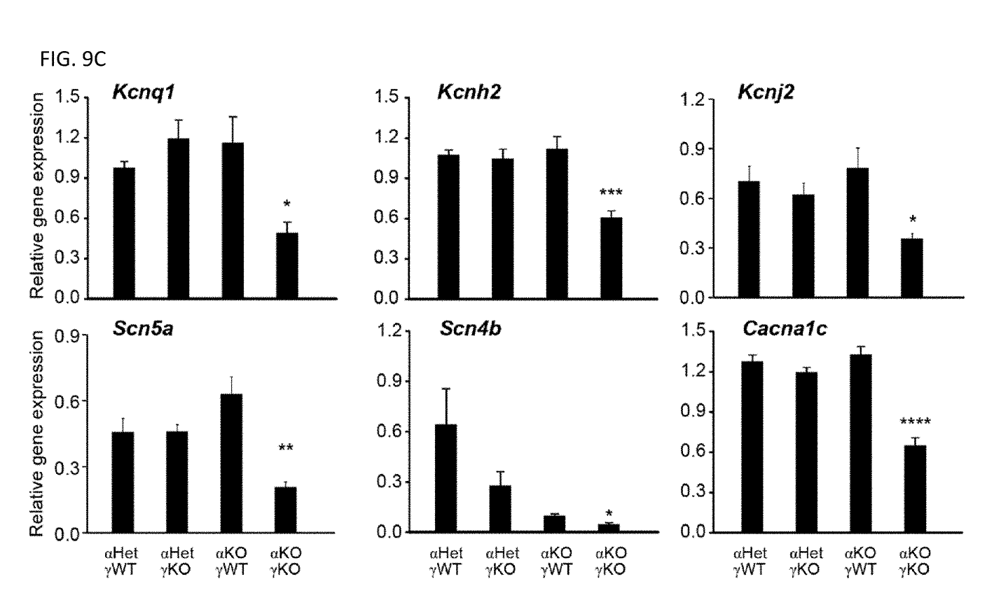

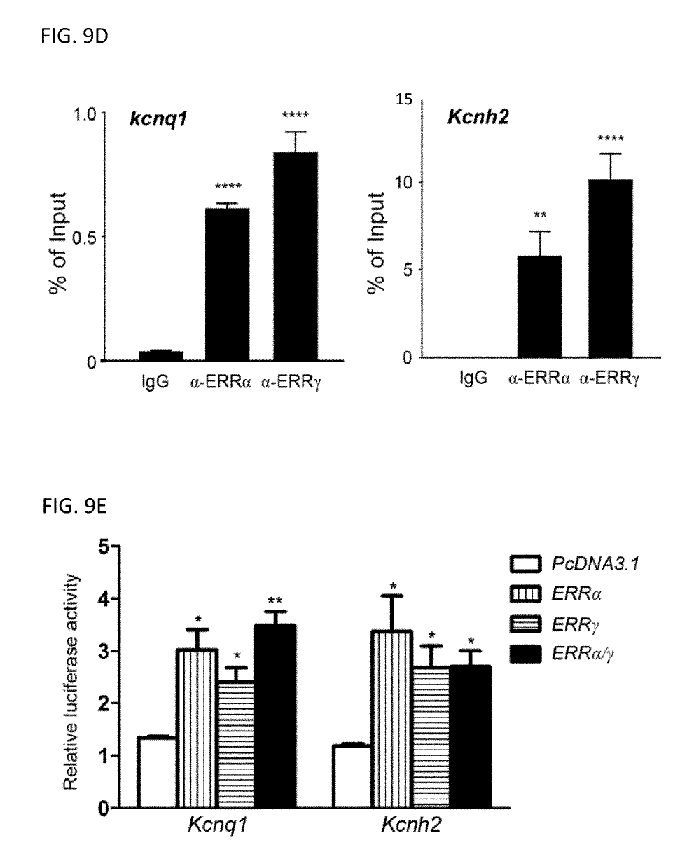

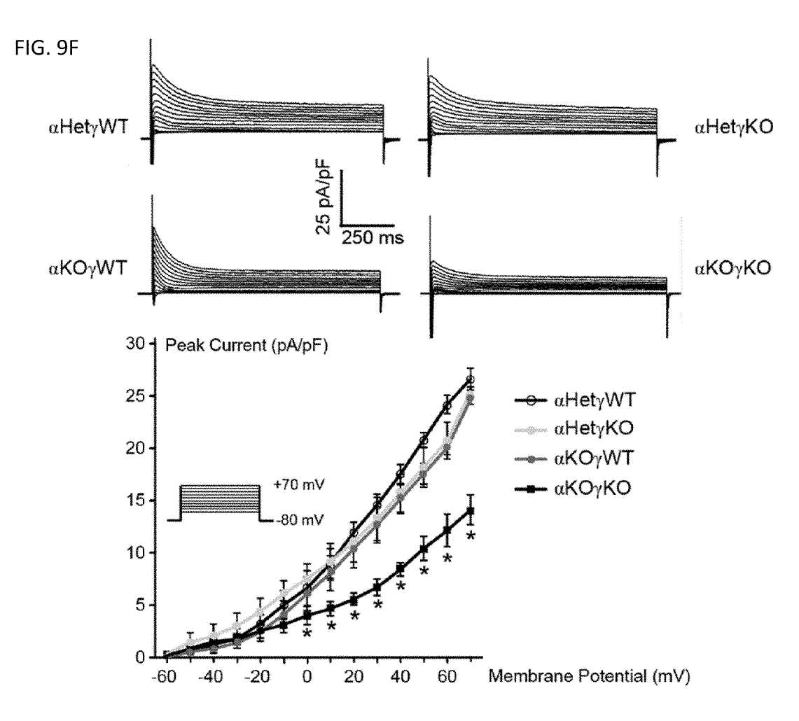

Statistical significance was calculated by one way ANOVA followed by Tukey's multiple comparison test (FIG. 7), two-tailed ANOVA (FIG. 9F) or two-tailed, unpaired, unequal variance t-test (the rest of the figures) and was indicated when p value reached less than 0.05.

### Example 2

### Mice Lacking Both ERRα and ERRγ in the Heart Die Postnatally with Cardiomyopathy

The whole body ERRα KO mice exhibit no cardiac defects under normal, unstressed conditions (Huss et al., 2007. Cell Metab 6:25-37). The whole body ERRγ KO mice die within 48 hours after birth (Alaynick et al., 2007. Cell Metab 6:13-24), thus preventing the investigation of ERRγ function in the postnatal heart. To circumvent this early lethality, cardiac-specific ERRγ KO (ERRγ^{flox/flox}Cre⁺) mice were generated by crossing mice possessing floxed ERRγ alleles with Myh6-Cre mice (Agah et al., 1997. J Clin Invest 100:169-179). All mice were backcrossed at least 5-6 generations to and maintained in the C57BL6/J background.

qRT-PCR and western blot analysis confirmed the almost complete loss of ERRγ RNA and protein in their hearts but not in other abundantly-expressed tissues (brain, kidney, brown adipose tissue and soleus) (FIGS. 1A and B), consistent with the reported cardiac-specific recombination mediated by Myh6-Cre. Expression of ERRα and ERRβ was not significantly changed in any of these tissues including the heart (FIG. 1A). The cardiac-specific ERRγ KO mice showed normal survival (FIG. 1C), appeared normal and displayed no cardiac or other abnormalities at least during the first month of life (see below). Therefore the lethality phenotype of the whole body ERRγ KO mice was not due to the loss of cardiac ERRγ itself.

These cardiac-specific ERRγ KO (hereafter referred as αWTγKO for simplicity) mice were further bred with whole body ERRα KO mice to generate mice lacking both ERRα and ERRγ in the heart (ERRα^{-/-}ERRγ^{flox/flox}Cre⁺ or αKOγKO). Both ERRα and ERRγ RNA and protein were barely detectable in the hearts of the αKOγKO mice by 3 days of age (FIG. 1B). The αKOγKO mice were born at close to the predicted Mendelian ratio. However, the αKOγKO pups exhibited early postnatal lethality and none of them survived past the first month of life, with most of them dying between 13 and 26 days of age (FIG. 1C). In contrast all littermates lacking 0-3 alleles of ERRα or ERRγ (αWTγWT−ERRα^{+/+}ERRγ^{flox/flox}Cre⁻, αWTγKO−ERRα^{+/+}ERRγ^{flox/flox}Cre⁺, αHetγWT−ERRα^{+/-}ERRγ^{flox/flox}Cre⁻, αHetγKO−ERRα^{+/-}ERRγ^{flox/flox}Cre⁺, and αKOγWT−ERRα^{-/-}ERRγ^{flox/flox}Cre⁻) had normal survival rate. Due to this early lethality of αKOγKO pups and the fact that no physiological or phenotypical difference between ERRα WT and heterozygous (Het) mice was observed by us nor previously reported (21, 23), αHetγKO with αKOγWT mice were bred to generate αHetγWT, αHetγKO, αKOγWT and αKOγKO littermates (1:1:1:1 expected ratio, FIG. 1D) for all the following studies.

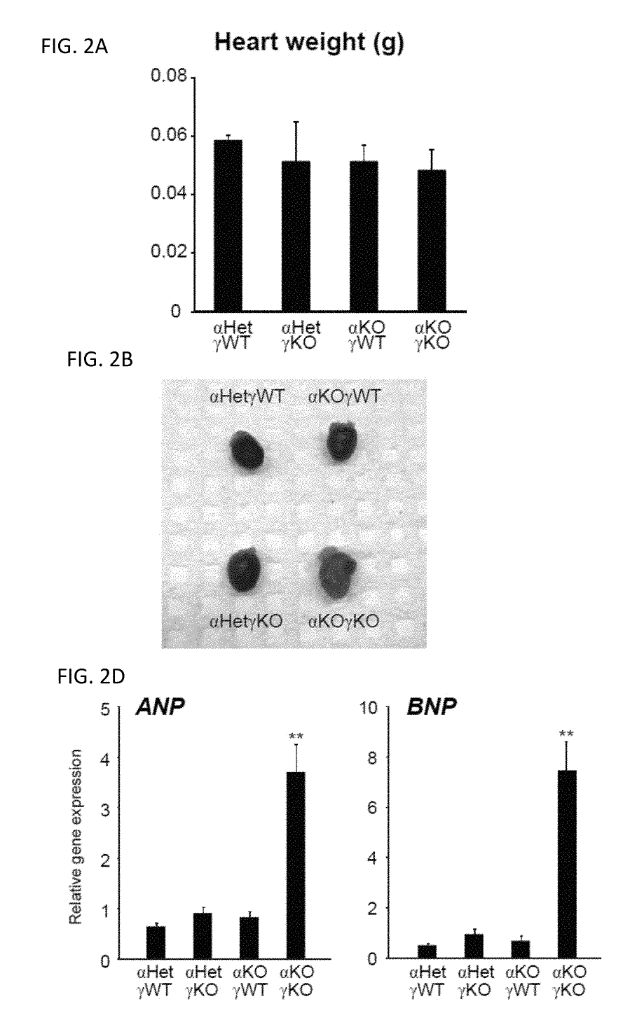

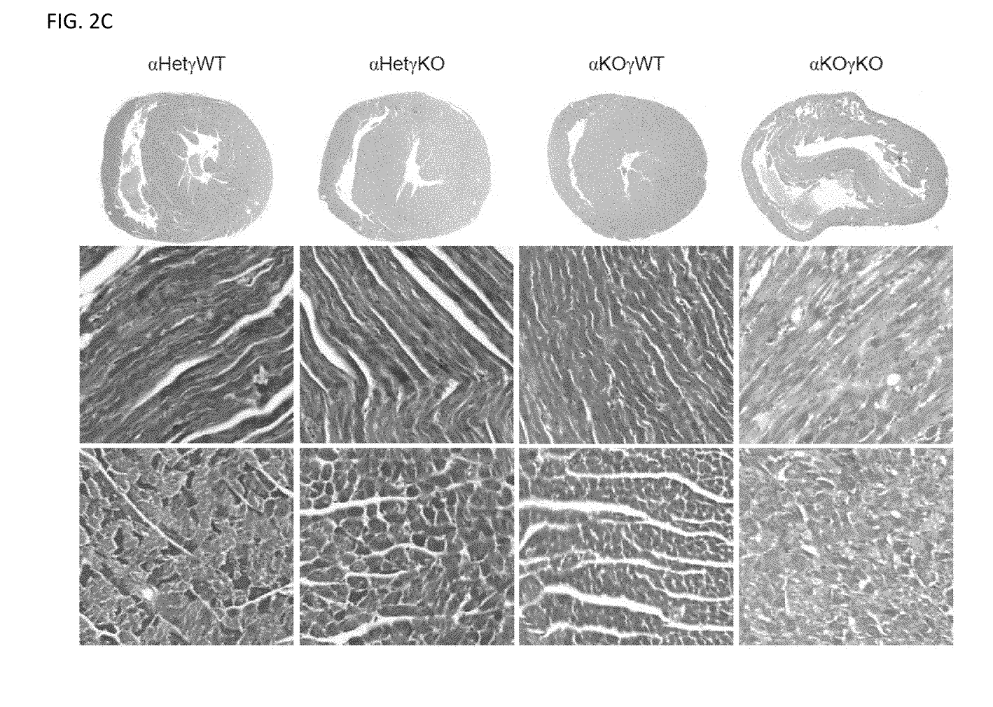

Since loss of ERRγ in the whole body ERRα KO background was cardiac-specific and only αKOγKO pups exhibited this postnatal lethality, this study focused on the hearts of these mice. Their heart weight and left ventricles (LV) mass were comparable to their littermates at 16 days of age (FIG. 2A, and see below in FIG. 7B). Compared to control αHetγWT hearts, normal histology in αHetγKO and αWTγKO mouse hearts was observed. In contrast, αKOγKO mouse hearts exhibited features of developing dilated cardiomyopathy, including enlargement of both ventricles but no significant changes in the ventricle wall and septum thickness (FIGS. 2B and C; also see below in FIG. 7). In support of these histological observations, expression of ANP and BNP, markers associated with cardiomyopathy and heart failure (Boomsma et al., 2001. Cardiovasc Res 51:442-449; Maisel 2001. Cardiol Clin 19:557-571), were significantly elevated in αKOγKO hearts (FIG. 2D).

These results reveal the essential role of ERRα and ERRγ in postnatal cardiac health.

### Example 3

### ERRα and ERRγ Regulate Cardiomyocyte Metabolism

FAO and the ensuing OxPhos in the mitochondria provide a major source of energy needed for adult myocardium. Previous ChIP-on-Chip studies revealed that promoters of many cardiac FAO and OxPhos genes were bound by both ERRα and ERRγ, establishing them as direct transcriptional targets of ERRα and ERRγ (Dufour et al., 2007. Cell Metab 5:345-356). However, expression of only some of these genes was changed in the hearts of adult whole body ERRα KO or neonatal whole body ERRγ KO mice. In addition, it was not determined whether OxPhos or other mitochondrial functions were changed in these single ERRα or ERRγ KO mouse hearts, considering the significant overlap of target genes between ERRα and ERRγ.

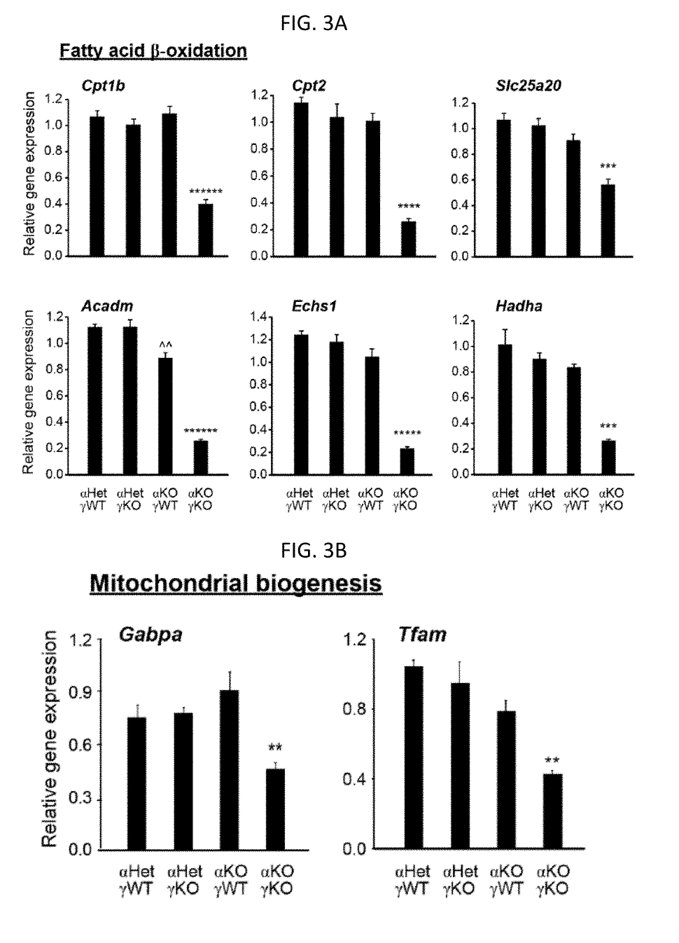

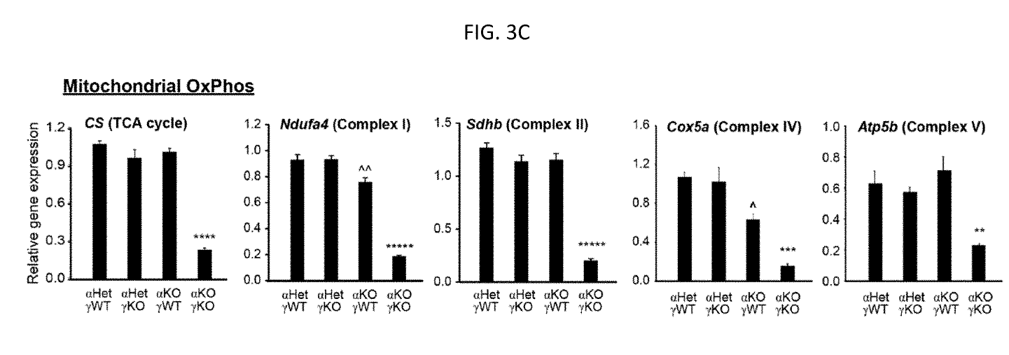

qRT-PCR was used to examine the expression of genes important in cardiac metabolism in the αKOγKO hearts. It was observed that expression of a large number of genes was significantly decreased compared to littermate controls. These included known and previously unknown ERR target genes and included almost all genes in the mitochondrial FAO pathway (Cpt1b, Cpt2, Slc25a20, Acadm, Echs1, and Hadha, FIG. 3A), genes important in the transcriptional control of mitochondrial biogenesis (Gabpa and Tfam, FIG. 3B), as well as over 40 genes encoding proteins of the mitochondrial tricarboxylic acid (TCA) cycle and electron transport chain (ETC) complexes, a subset of which are shown in FIG. 3C (Citrate synthase (CS), Ndufa4, Sdhb, Cox5v, and Atp5b).

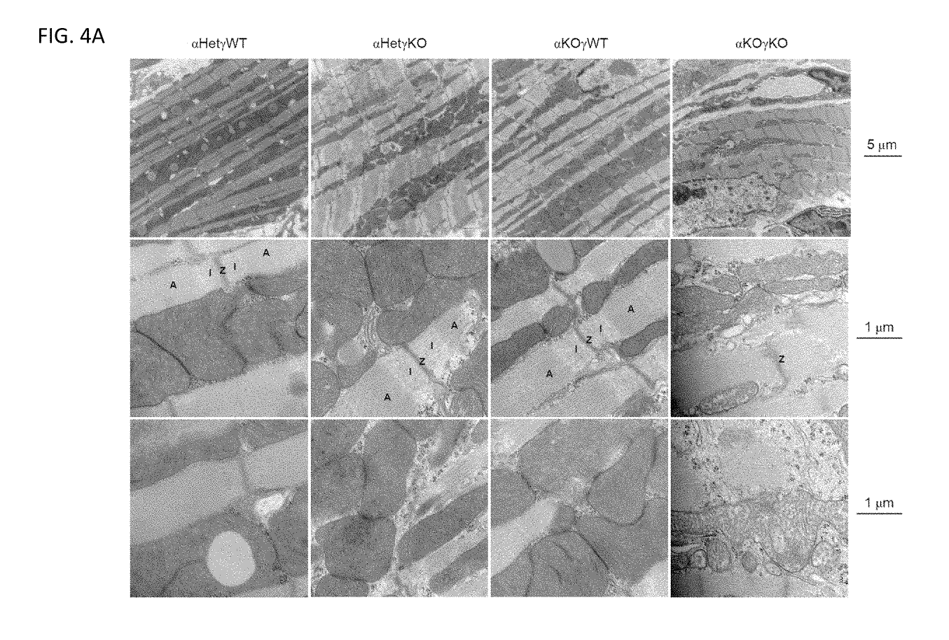

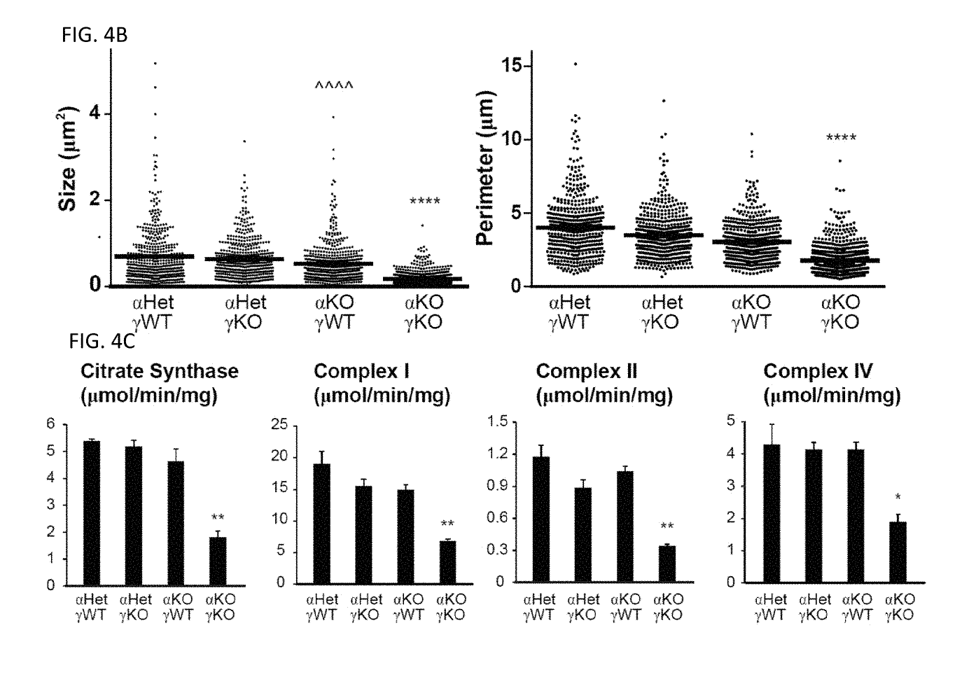

The mitochondrial morphology was evaluated using EM. Previous studies found no structural changes of mitochondria and sarcomeres in whole body single ERRα or ERRγ KO mouse hearts. Consistent with these reports and the gene expression studies (FIG. 3), no defects were found in cardiac mitochondrial and sarcomeric ultrastructures in all genotypes except the αKOγKO hearts (FIG. 4A). The αKOγKO hearts exhibited several notable ultrastructural abnormalities including distorted myofibrils (FIG. 4A, top row) consistent with histological observations (FIG. 2C). In particular, compared to the cardiac sarcomeres of control littermates displaying clear A and I bands, many αKOγKO heart sarcomeres seemed to lack clear boundaries between the A band and I band (FIG. 4A, middle row). In contrast to well-organized, densely packed mitochondria along the myofibrils in hearts of littermate controls, αKOγKO heart mitochondria appeared severely fragmented and showed significant loss of matrix density and clear cristae membranes (FIG. 4A, middle and bottom rows). In support of this observation of mitochondria fragmentation, αKOγKO heart mitochondria were significantly smaller as quantified by their size and perimeter (FIG. 4B). In line with these gene expression and structural changes, only the αKOγKO hearts exhibited significant loss of mitochondrial functions essential for energy generation, including decreased enzymatic activities of TCA cycle (citrate synthase) and ETC complexes (FIG. 4C). These results indicate that ERRα and ERRγ together are essential transcriptional regulators of cardiomyocyte metabolism.

### Example 4

### ERRα and ERRγ are Important for Integral Mitochondrial Dynamics

Mitochondria inside a healthy cell form a dynamic network, which is essential for mitochondrial quality control by constantly fusing and dividing to exchange contents. In addition, mitochondrial fusion and fission are important and conserved mechanisms from yeast to mammals that ensure integral mitochondrial function, balancing cellular energy demand and supply, apoptosis, and other cellular functions. Mutation of genes involved in mitochondrial fusion and fission cause a variety of diseases including cardiomyopathy, optic atrophy and axonal neuropathy in humans and other animals.

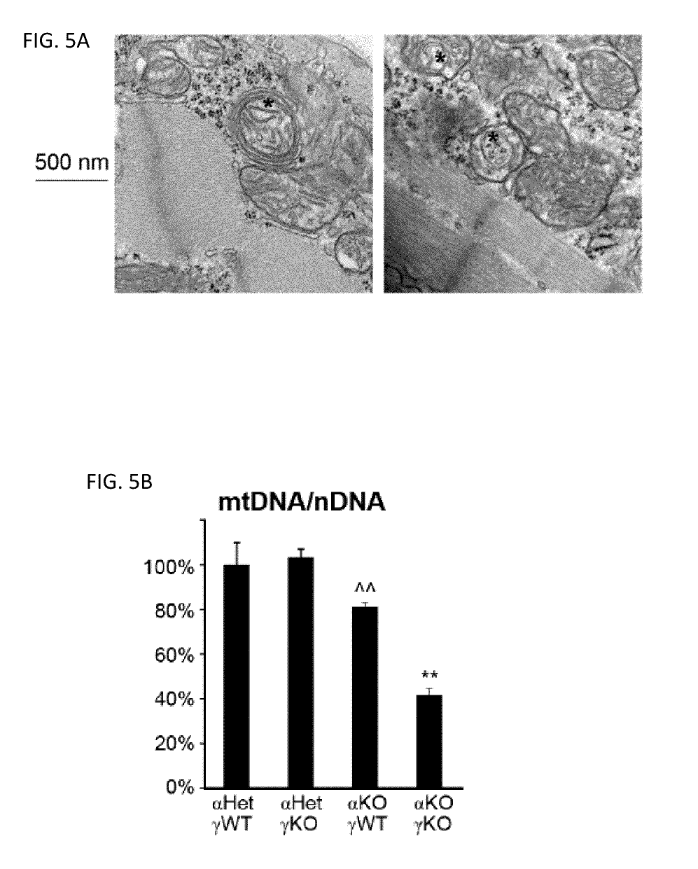

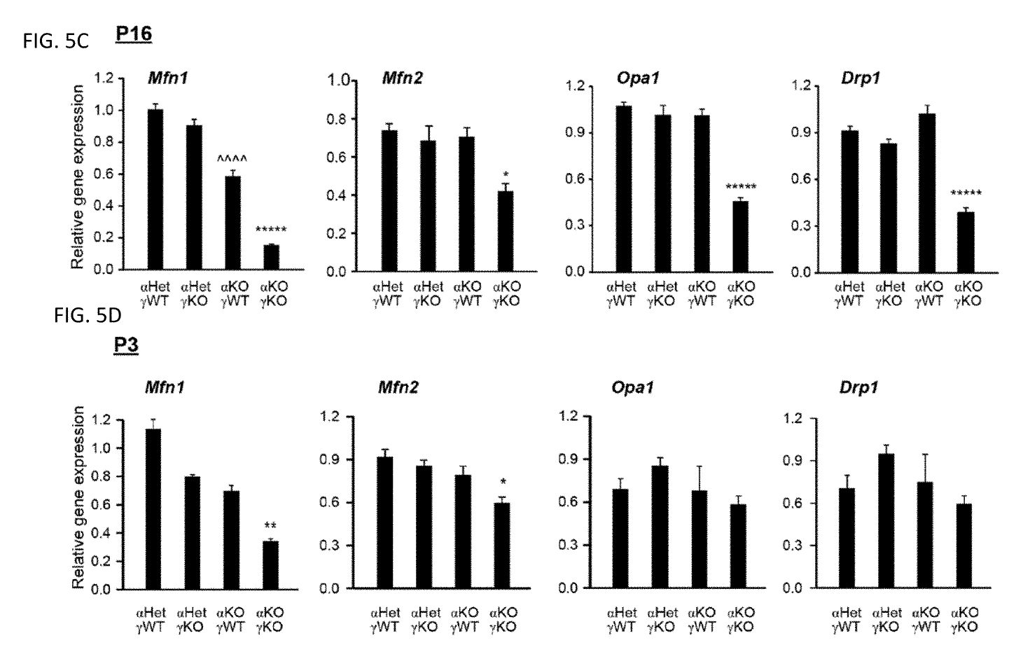

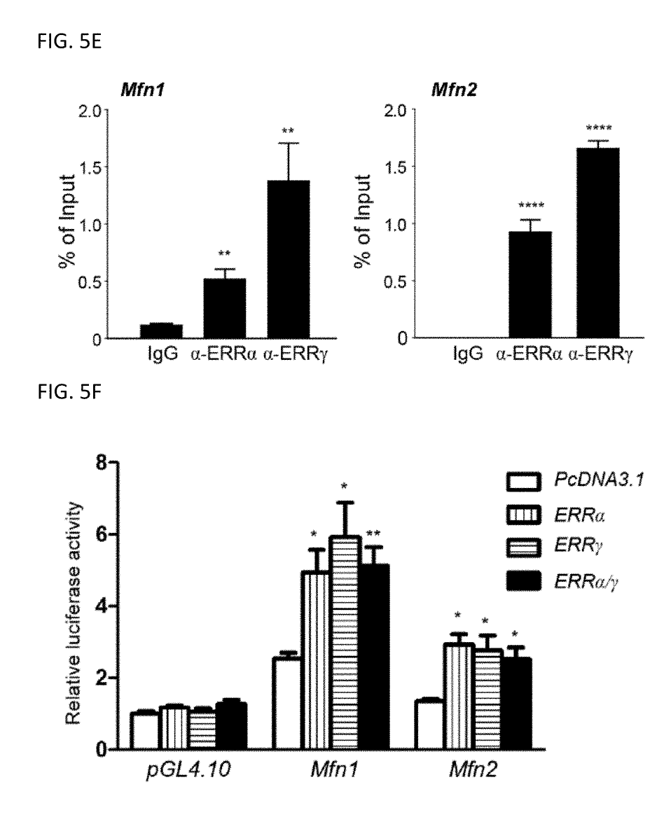

It was observed that the mitochondrial fragmentation and other ultrastructural defects in αKOγKO hearts were similar to those observed in mitochondrial dynamics-defective animal models (Chen et al., 2010. Cell 141:280-289; Papanicolaou et al., 2012. Circ Res 111:1012-1026; Chen et al., 2011. Circ Res 109:1327-1331; Otera et al., 2010. J Cell Biol 191:1141-1158; Martin et al., 2013. Circ Res. 114:626-36). Some mitochondria in the αKOγKO hearts were wrapped by multiple double membranes (FIG. 5A), indicating mitochondrial dynamics defects. Consistent with the notion that mitochondrial dynamics is essential to maintain mitochondrial DNA (mtDNA) stability and quantity, mtDNA amount decreased by about 60% in the αKOγKO hearts (FIG. 5B). Accordingly, expression of almost all genes essential for mitochondrial fusion and fission were significantly reduced in 16 day old αKOγKO hearts (FIG. 5C). These included critical mitochondrial fusion genes Mfn1, Mfn2 and Opa1 as well as key mitochondrial fission gene Drp1. Expression of Mfn1 and Mfn2 (but not Opa1 and Drp1) was also significantly decreased in αKOγKO hearts at a much younger age (3 day old, FIG. 5D), indicating they are likely direct transcriptional targets of ERRα and ERRγ. Conserved ERR response elements (ERRE) were found within the first intron of the mouse Mfn1 gene and in the promoter of the mouse Mfn2 gene. Chromatin immunoprecipitation (ChIP) assay confirmed that ERRα and ERRγ directly bound to these ERREs (FIG. 5E). Furthermore, both ERRα and ERRγ activated these ERRE-driven luciferase reporters (FIG. 5F), establishing Mfn1 and Mfn2 as direct ERRα and ERRγ target genes in the heart.

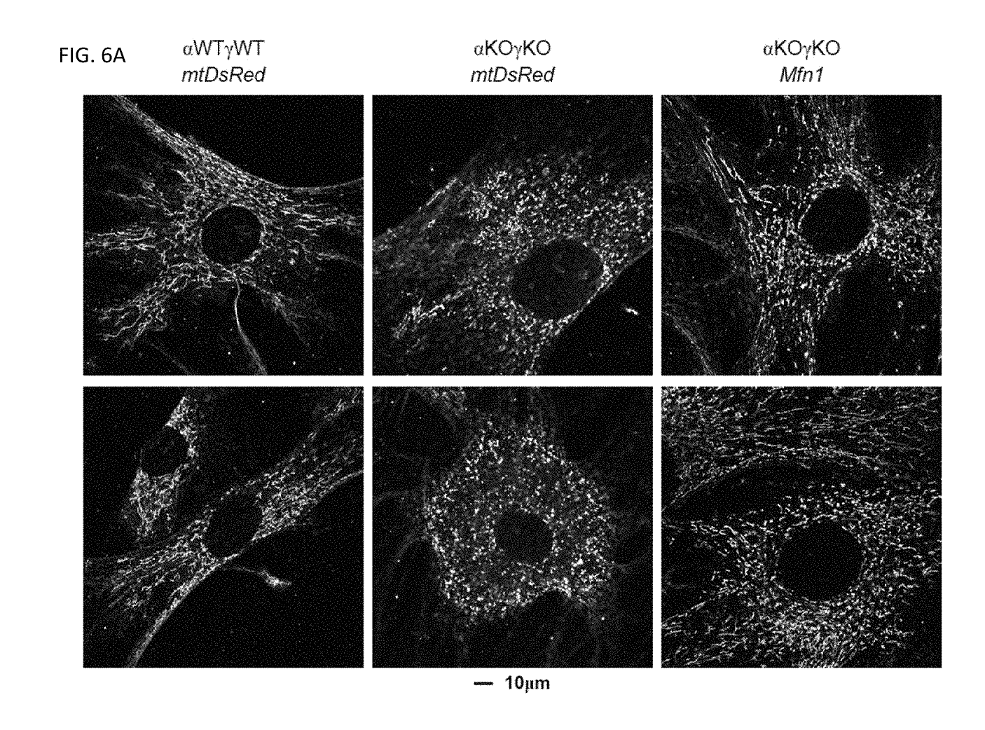

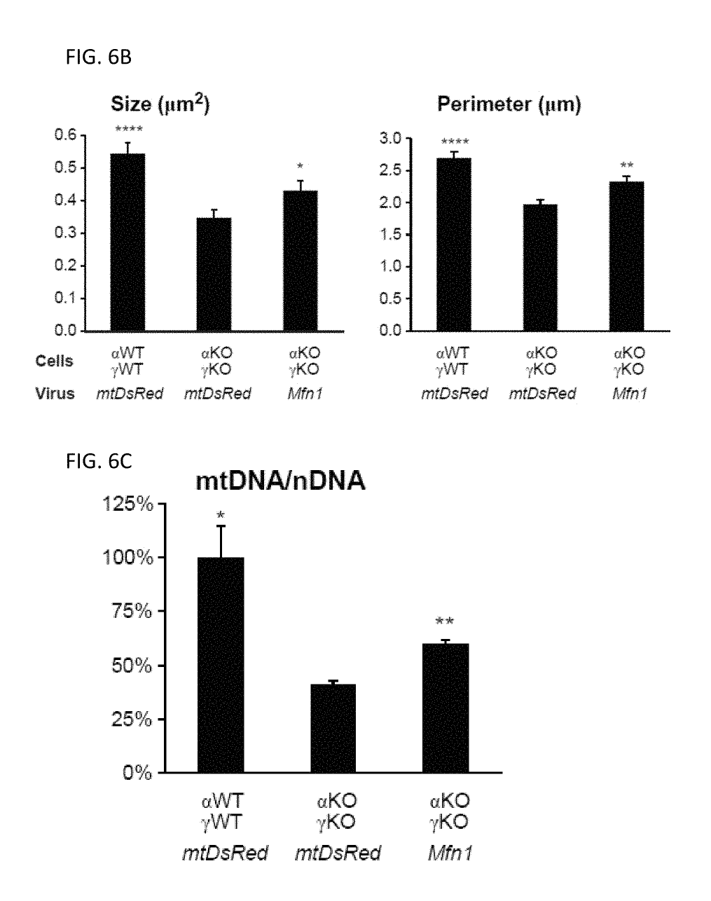

It was determined whether the reduced level of these genes was responsible for the mitochondrial dynamics defects and mtDNA loss in the αKOγKO hearts. The αKOγKO cells in vitro recapitulated the mitochondrial defects in vivo, including fragmented mitochondria (FIGS. 6A and B compared to FIGS. 4A and B) and loss of mtDNA (FIG. 6C compared to FIG. 5B). It was then determined whether restored expression of these mitochondrial dynamics genes would rescue the mitochondrial phenotype. Overexpression of Mfn1 alone significantly alleviated mitochondrial fragmentation (FIG. 6A) as quantified by their size and perimeters (FIG. 6B) and the mtDNA quantity (FIG. 6C) in the αKOγKO cells. The rescue was not complete, which could be because expression of other genes important in mitochondrial dynamics (FIG. 5) and mtDNA quantity (such as Tfam, FIG. 3B) were not restored, or due to incomplete retroviral infection (about 70-80% cells were infected judged by mtDsRed). These results demonstrate the important role of ERRα and ERRγ in controlling mitochondrial dynamics.

### Example 5

### ERRα and ERRγ are Vital for Cardiac Contractile Function

Previous ChIP-on-Chip and gain-of-function studies have found that ERRα and ERRγ directly regulate the expression of cardiac contractile genes including Myh6, Acta1, Tnni3, Phospholamban (Pln) and Atp2a2 (also known as Serca2) (Dufour et al., 2007. Cell Metab 5:345-356). However, it is unclear whether ERRα and ERRγ are absolutely required for their expression in a loss-of-function context as expression of these genes was not or only slightly changed in ERRα KO or ERRγ KO hearts at basal states. More importantly, no cardiac contractile defects were previously observed in these ERRα KO or ERRγ KO hearts at basal states.

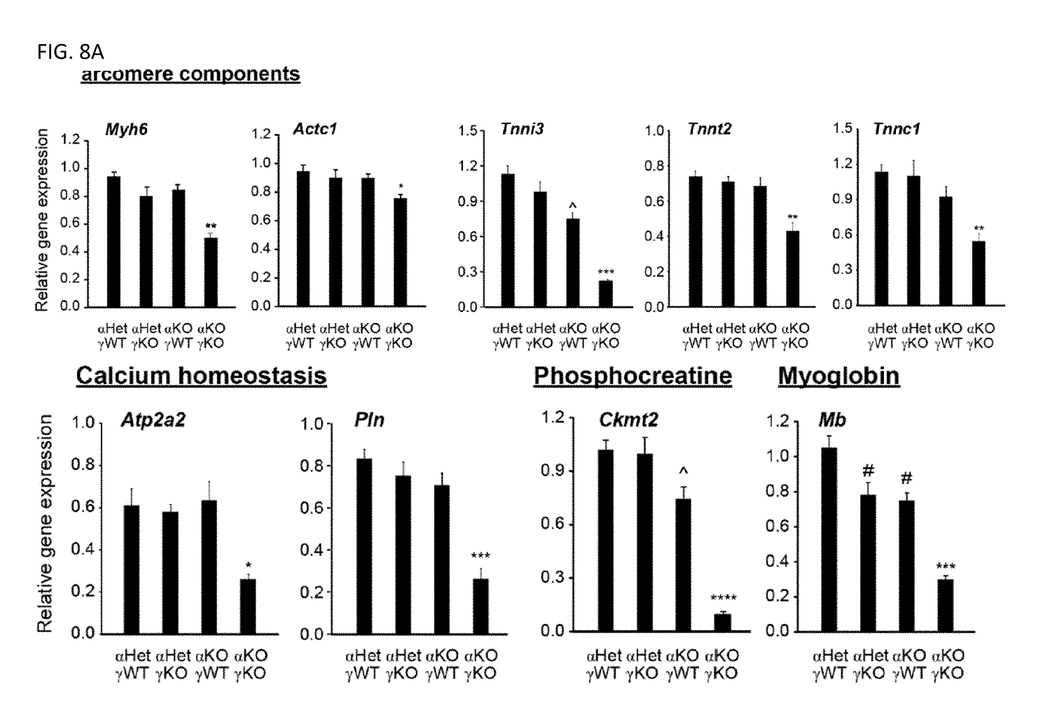

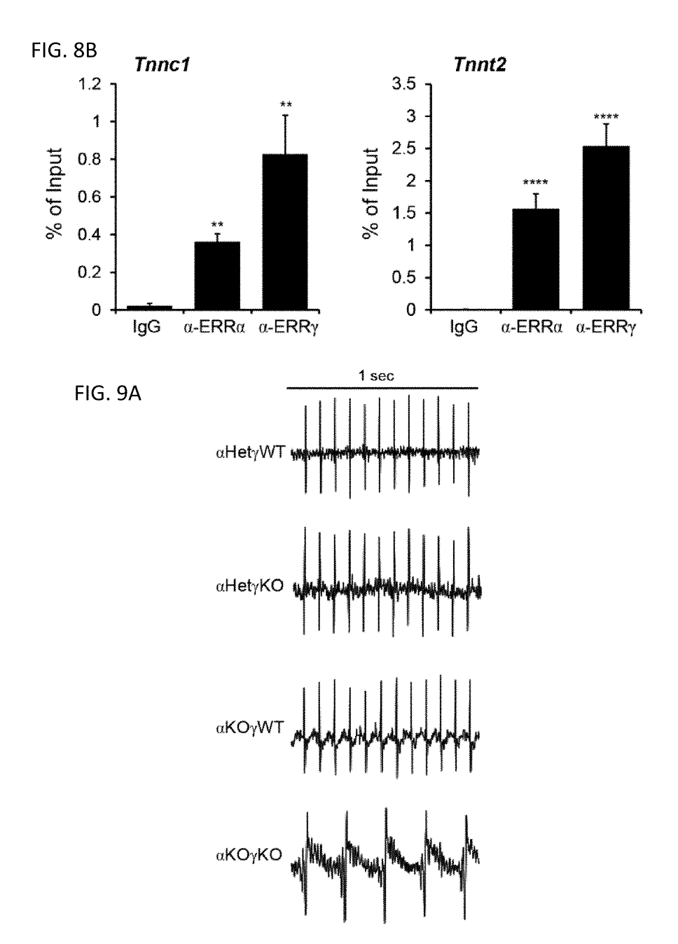

Echocardiography was used to determine the cardiac contractile function in αKOγKO mice and their littermates (FIG. 7A). Although the absolute and relative (normalized to body weight) LV mass, absolute and relative (normalized to LV total dimension) LV wall thickness (FIG. 7B), diastolic LV dimension (LVIDd) and volume (LV EDV) remained similar among all genotypes (FIG. 7C), the systolic LV dimension (LVIDs) and volume (LV ESV) were significantly increased in αKOγKO hearts but not in hearts lacking either ERRα or ERRγ alone (FIG. 7C). These were consistent with the histological observations of developing dilated cardiomyopathy (FIG. 2C). More importantly, the αKOγKO hearts exhibited significantly decreased cardiac contractile function measured by ejection fraction (EF) and cardiac output (CO) (FIG. 7D). For example, compared to the 60% EF within normal range in the control animal hearts, αKOγKO hearts have an EF of only 20%, a value indicating severe cardiomyopathy or heart failure clinically. In line with these functional changes, αKOγKO hearts had significantly reduced expression of Myh6, Actc1, Tnni3, Mb, Atp2a2, Pln and Ckmt2, genes encoding proteins of sarcomere components, myoglobin, or important in calcium homeostasis and the phosphocreatine system (FIG. 8A).

Additional, previously unknown ERRα and ERRγ target genes important in cardiac contraction were identified. These include Tnnt2 and Tnnc1 whose expressions were decreased in αKOγKO hearts (FIG. 8A). Conserved ERREs were found located within the first intron of both Tnnt2 and Tnnc1 genes, and confirmed that ERRα and ERRγ directly bound to these ERREs by ChIP (FIG. 8B). Together, these studies establish that ERRα and ERRγ are crucial transcriptional regulators of cardiac contractile function.

### Example 6

### ERRα and ERRγ are Essential for Normal Myocardial Conduction Through Transcriptional Regulation of Key Potassium, Sodium and Calcium Channel Genes

In addition to contraction, electric conduction is another essential and major energy consuming cardiac function. Defects in heart rate, myocardial conduction and repolarization that reduce metabolic demand by the weakened myocardium are often associated with the onset of cardiomyopathy. Whole body ERRγ KO mice exhibit neonatal lethality and slightly prolonged QRS complex and QT intervals (Alaynick et al., 2010. Mol Endocrinol 24:299-309). However, it is shown herein that mice lacking cardiac ERRγ alone (in both ERRα WT and Het backgrounds) were viable with normal cardiac structure, metabolism, mitochondrial dynamics and contractile function (FIGS. 1-8). This raised the question of whether the long QT intervals and associated gene expression changes in the neonatal whole body ERRγ KO pups was due to cardiac autonomous defect, or impacted upon by loss of ERRγ in other tissues such as the brain. ECG was used to investigate this in conscious, ambulatory cardiac-specific ERRγ KO and αKOγKO pups. Compared to control αHetγWT littermates, we found no ECG defects in mice lacking either only ERRα (αKOγWT) or cardiac ERRγ (αHetγKO) (FIGS. 9A and B). Therefore cardiac ERRγ itself was not required for maintaining normal myocardial conduction.

In sharp contrast, mice lacking both ERRα and cardiac ERRγ (αKOγKO) exhibited abnormal ECG and severe bradycardia with heart rate only about half of their littermate controls (FIGS. 9A and B). This notable bradycardia phenotype was not seen in either αKOγWT or αHetγKO littermates, nor previously reported in whole body adult ERRα or neonatal ERRγ KO mice. In addition, the QRS complex and the PR and QT intervals were significantly prolonged in the αKOγKO hearts compared to all other genotypes (FIG. 9B).

In the setting of cardiomyopathy, transcriptional alterations that directly affect cardiac ion channel expression and function contribute to the electrophysiological remodeling. Therefore, the expression of important cardiac ion channel genes, especially those implicated in human conduction disorders, was examined. Expression of multiple cardiac potassium (Kcnq1, Kcnh2, Kcnj2), sodium (Scn5a, Scn4b) and calcium (Cacna1c) channel genes were significantly decreased in αKOγKO hearts compared to controls (FIG. 9C). This is clinically relevant as mutations of all these genes cause conduction disorders with similar symptoms including prolonged QT intervals in humans.

It was determined whether transcription of these ion channel genes was directly controlled by ERRα and ERRγ. Testing this for the genes encoding Kcnq1 and Kcnh2, two of the most abundantly expressed voltage-dependent potassium channel proteins essential for myocardial repolarization, it was observed that ERRα and ERRγ directly bound to a conserved region located in the first intron of both genes (FIG. 9D). In addition, ERRα and ERRγ activated these enhancers in a luciferase reporter assay (FIG. 9E). Acutely isolated αKOγKO and control ventricular cardiomyocytes were used to determine whether the potassium currents were altered. These studies revealed that peak global potassium currents were reduced by about 50% in αKOγKO cardiomyocytes compared to those isolated from hearts of control littermates (FIG. 9F), consistent with cardiomyocyte-autonomous defects of potassium channels. Taken together these data indicate that ERRα and ERRγ are essential transcriptional regulators for normal myocardial conduction.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A method of increasing cardiac contraction, increasing mitochondrial activity, and/or increasing oxphos activity, comprising:
administering a therapeutically effective amount of one or more agents that increases estrogen-related receptor (ERR) α activity to a mammal needing increased cardiac contraction, increased mitochondrial activity, and/or increased oxphos activity; and
administering a therapeutically effective amount of one or more agents that increases ERRγ activity to a mammal needing increased cardiac contraction, increased mitochondrial activity, and/or increased oxphos activity.

2. The method of claim 1, wherein the method further comprises not exercising the mammal.

3. The method of claim 1, wherein the method further comprises:
selecting a mammal in need of increased cardiac contraction, increased mitochondrial activity, and/or increased oxphos activity or a mammal at risk for developing a disorder that can benefit from increased cardiac contraction, increased mitochondrial activity, and/or increased oxphos activity.

4. The method of claim 1, wherein the mammal has or is at risk for heart failure, bradycardia and/or cardiomyopathy.

5. The method of claim 1, wherein the method further comprises administering to the mammal a therapeutically effective amount of an agent that balances electrolytes, an agent that prevents arrhythmias, an agent that lowers blood pressure, an agent that prevents blood clots from forming, an agent that reduces inflammation, an agent that removes excess sodium, an agent that decreases heart rate, isosorbide dinitrate/hydralazine hydrochloride, or combinations thereof.

6. The method of claim 1, wherein the method further comprises administering a therapeutically effective amount of one or more agents that increases mitofusin 1 (Mfn1) activity to a mammal needing increased cardiac contraction, increased mitochondrial activity, and/or increased oxphos activity.

7. The method of claim 1, wherein the mammal cannot exercise or is sedentary.

8. The method of claim 1, wherein the mammal is a human.

9. The method of claim 1, wherein the administration comprises parenteral, subcutaneous, intraperitoneal, intrapulmonary, or intranasal administration.

10. A kit for increasing cardiac contraction, increasing mitochondrial activity, and/or increasing oxphos activity, comprising:
one or more agents that increases ERRα activity; and
one or more agents that increases ERRγ activity.

11. The kit of claim 10, wherein the kit further comprises one or more agents that increases Mfn1 activity.

12. The method of claim 1, wherein the one or more agents that increases ERRγ activity comprises:
a nucleic acid molecule encoding ERRγ;
one or more ERRγ agonists;
an ERRγ protein; or
combinations thereof.

13. The method of claim 1, wherein the one or more agents that increases ERRγ activity is: 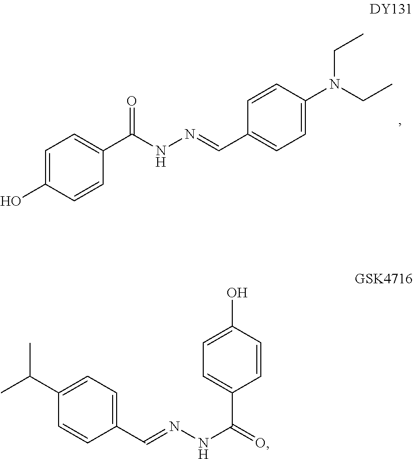
or combinations thereof.

14. The method of claim 1, wherein the one or more agents that increases ERRγ activity is:

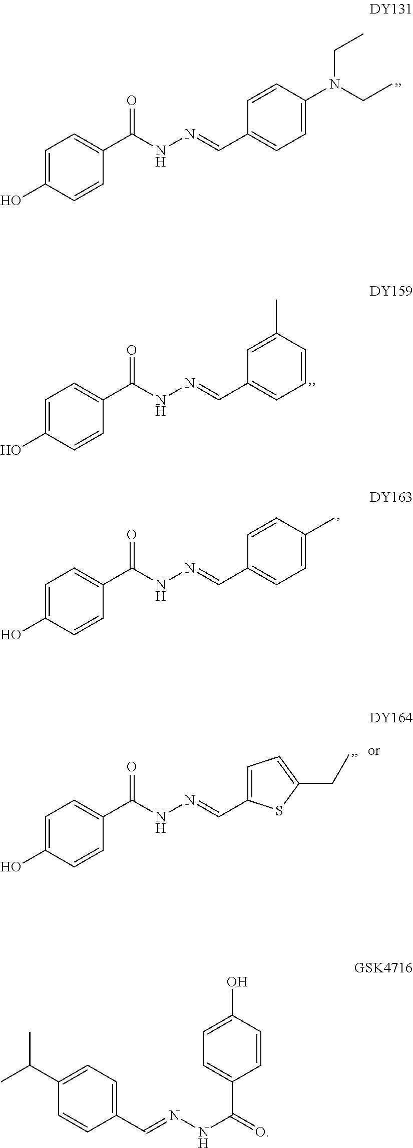

15. The method of claim 1, wherein the one or more agents that increases ERRγ activity is: 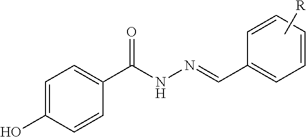
wherein R is H (DY162), p-CH₃ (DY163), 2-Cl, 3-CF₃ (DY165), p-CF₃ (DY168), p-OCH₃ (DY169), 3-NO₂, 4CF₃ (DY170), 2,3-O₂CH₃ (DY174), or m-CH₃ (DY159), 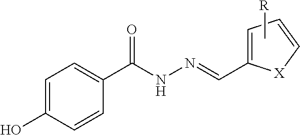
wherein X is S and R is 5-CH₃ (DY166), 5-CH₂CH₃ (DY164), or 5-NO₂ (DY167); wherein X is O and R is 4,5-CH₃ (DY173) or CH₂CH₃ (DY175), or wherein X is CH, and R is 2-Cl, 3-CF₃, p-CF₃; p-OCH₃, 3-NO₂, 4-CF₃; or 2,3-O₂CH₃; 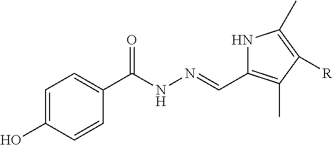
wherein R is H (DY117) or R is Br (DY172), 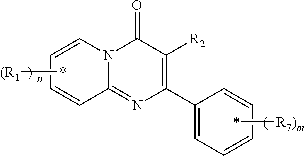
wherein
m is 0, 1 or 2;
n is 0, 1 or 2;
R₁ and R₇ are independently selected from
1) H;
2) Halo;
3) OH;
4) (C═O)ₐ, O_{b}C₁-C₄ alkyl, wherein a is 0 or 1 and b is 0 or 1, wherein the alkyl can be substituted by 0, 1 or more substituted groups independently selected from H or C₃C₆ heterocyclyl;
5) (C═O), O_{b}C₃-C₆ cycloalkyl, wherein a is 0 or 1 and b is 0 or 1;
R2 is selected from:
1) H;
2) C₁-C₃ alkyl, wherein the alkyl can be substituted by 0, 1 or more substituted groups independently selected from H or C₃C₆ heterocyclyl;
3) C₃-C₆ cycloalkyl; 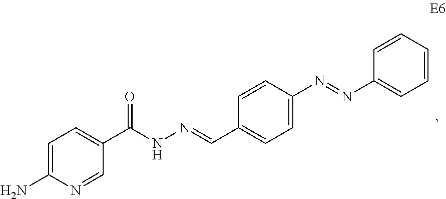 [SMILES: Nc1ccc(C(=O)N/N=C/c2ccc(/N=N/c3ccccc3)cc2)cn1]
or combinations thereof.

16. The method of claim 1, wherein the one or more agents that increases ERRα activity comprises:
a nucleic acid molecule encoding ERRα;
one or more ERRα agonists;
an ERRα protein; or
combinations thereof.

17. The method of claim 1, wherein the one or more agents that increases ERRα activity comprises: 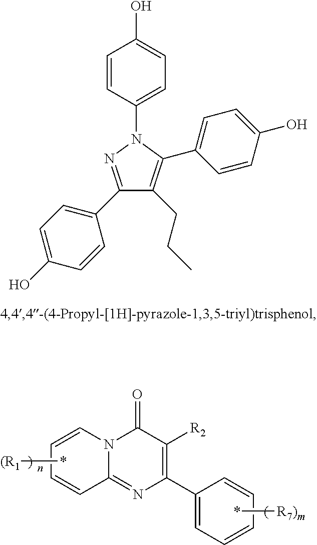
wherein:
m is 0, 1 or 2;
n is 1 or 2;
in the following, a is 0 or 1, b is 0 or 1;
each occurrence of R₁ is independently selected from the group consisting of:
1) Halo;
2) OH;
3) (C══O)ₐO_{b}C₁-C₄ alkyl; and
4) (C══O)ₐO_{b}C₃-C₆ cycloalkyl,
wherein one occurrence of R₁ is at the 8-position of the pyrido [1,2-a]pyrimidin-4-one ring;
each occurrence of R₂ is independently selected from the group consisting of:
1) Halo;
2) OH;
3) (C══O)ₐO_{b}C₁-C₄ alkyl, wherein, if a is 0 and b is 1, the alkyl is substituted with C₃-C₆ heterocyclyl; or
4) (C══O)ₐO_{b}C₃-C₆ cycloalkyl;
wherein, if one occurrence R₇ is halo of C₁-C₄ alkyl, at least one occurrence of R₁ is OH or (C══O)ₐO_{b}C₁-C₄ alkyl wherein a is 0 and b is 1;
R₂ is selected from the group consisting of:
1) H;
2) C₁-C₃ alkyl; and
3) C₃-C₆ cycloalkyl;
the alkyl mentioned above can be substituted by 0, 1 or more substituted R₄ groups wherein R₄ is selected from the group consisting of:
1) H; and
2) C₃-C₆ heterocyclyl,
or combinations thereof.

18. The method of claim 6, wherein the one or more agents that increases Mfn1 activity comprises:
a nucleic acid molecule encoding Mfn1;
one or more Mfn1 agonists;
an Mfn1 protein; or
combinations thereof.
